SEQUENCE LISTING (16 sequences)

SEQ ID NO: 1 - protein, 10 aa, synthetic construct
  REGION 1..10: note = Synthetic
GPGPGPGPGP

SEQ ID NO: 2 - protein, 10 aa, synthetic construct
  REGION 1..10: note = Synthetic
GAGAGAGAGA

SEQ ID NO: 3 - protein, 10 aa, synthetic construct
  REGION 1..10: note = Synthetic
GRGRGRGRGR

SEQ ID NO: 4 - protein, 10 aa, synthetic construct
  REGION 1..10: note = Synthetic
PRPRPRPRPR

SEQ ID NO: 5 - protein, 11 aa, synthetic construct
GRERGEESLCF

SEQ ID NO: 6 - protein, 54 aa, synthetic construct
GRERGEPLLLSQPHIYTFGLSRNSLPLSPPSHSRPPGRGSHPPLEAGRGPHSMS

SEQ ID NO: 7 - protein, 44 aa, synthetic construct
GRERGEAFAFKPATYLHLWSVQKQPATFPSKPLPSPRQRFTSTP

SEQ ID NO: 8 - protein, 39 aa, synthetic construct
PLLSSPVSVSLSLSLRSPSHAEPKLDCTAAISAHCNLPA

SEQ ID NO: 9 - protein, 41 aa, synthetic construct
PLLSSPTSLRDSPASVCRAATPDWFWWRRGFAVLAGPVSSP

SEQ ID NO: 10 - protein, 43 aa, synthetic construct
PLLSSPPCVILLPQSAAPPRLTGFGGDGVSLCWPGQSPAPNRE

SEQ ID NO: 11 - protein, 9 aa, synthetic construct
ARERDSLCF

SEQ ID NO: 12 - protein, 43 aa, synthetic construct
GEGQPLLLSQPHIYTFGLSRNSLPLSPPSQSSPSSSTLAQRDS

SEQ ID NO: 13 - protein, 59 aa, synthetic construct
GRGTAFAFKPATYLHLWSVQKQPATFPSKPVITFIINISTELLIIVLTLCTMDPQLGWL

SEQ ID NO: 14 - protein, 30 aa, synthetic construct
MPLPRAHAATPDWFWWRRGFAVLAGQVSSP

SEQ ID NO: 15 - protein, 29 aa, synthetic construct
LSLAPRSPSHAEPKLDCTAAISAHCNLPA

SEQ ID NO: 16 - protein, 47 aa, synthetic construct
PSPHGLPLMRSQSWTVLLPSRLTATSLPDSPASVCRMPAIAGTRRHA

(19) United States
(12) Patent Application Publication
(10) Pub. No.: US 2025/0164489 A1
(43) Pub. Date: May 22, 2025

(54) RAN PROTEINS IN SPORADIC AMYOTROPHIC LATERAL SCLEROSIS

(71) Applicant: University of Florida Research Foundation, Incorporated, Gainesville, FL (US)

(72) Inventors: Laura Ranum, Gainesville, FL (US); Lien Nguyen, Gainesville, FL (US); Shu Guo, Gainesville, FL (US)

(73) Assignee: University of Florida Research Foundation, Incorporated, Gainesville, FL (US)

(21) Appl. No.: 18/841,754

(22) PCT Filed: Feb. 27, 2023
(86) PCT No.: PCT/US2023/063328

§ 371 Date: Aug. 27, 2024

Related U.S. Application Data
(60) Provisional application No. 63/314,981, filed on Feb. 28, 2022.

(51) Int. Cl. G01N 33/573; G01N 33/68

(52) U.S. Cl. CPC G01N 33/573; G01N 33/6896; G01N 2333/4719; G01N 2333/914; G01N 2800/2835

(57) ABSTRACT

Aspects of the disclosure relate to compositions and methods for the diagnosis and/or treatment of C9orf72 negative sporadic amyotrophic lateral sclerosis (C9− sALS). In some embodiments, the disclosure relates to identifying a subject as having C9orf72 negative (C9−) sALS by detecting expression or activity of repeat-associated non-ATG (RAN) translation proteins (e.g., RAN proteins). In some embodiments, the methods and compositions of the disclosure identify certain gene or genes which comprise mutation(s) leading to the expression of the detected RAN proteins, and which were previously unknown to be associated with sALS. In some embodiments, said gene(s) can be used to identify or diagnose subjects having, suspected of having, or at risk of developing sALS which is unrelated to expansion mutations within the C9orf72 and/or SCA36 genetic loci (e.g., C9− sALS). In some embodiments, the disclosure relates to methods of treating C9− sALS by administering to a subject in need thereof an agent that reduces expression or activity of RAN proteins.

### RELATED APPLICATIONS

This application claims the benefit under 35 U.S.C. § 119 (e) of U.S. provisional Application Ser. No. 63/314,981, filed Feb. 28, 2022, entitled “RAN PROTEINS IN SPORADIC AMYOTROPHIC LATERAL SCLEROSIS,” the entire contents of which are incorporated by reference herein.

### FEDERALLY SPONSORED RESEARCH

This invention was made with government support under grant numbers R01 NS098819 and R37 NS040389, awarded by the National Institutes of Health. The government has certain rights in the invention.

### REFERENCE TO AN ELECTRONIC SEQUENCE LISTING

The contents of the electronic sequence listing (U120270088WO00-SEQ-KZM.xml; size: 16,745 Bytes; and date of creation: Feb. 27, 2023) is herein incorporated by reference in its entirety.

### BACKGROUND

Microsatellite repeat expansions are known to cause more than forty neurodegenerative disorders. Molecular features common to many of these disorders include the accumulation of RNA foci containing sense and antisense expansion transcripts and the accumulation of proteins from repeat-associated non-AUG (RAN) translation. RAN translation can occur across a broad range of repeat lengths from pre-mutation lengths (˜30-40 repeats) to full expansions (up to 10,000 repeats). While repetitive elements account for a large portion of the human genome, the detection of repeats and repeat expansion mutations is challenging.

### SUMMARY

Several diseases, including amyotrophic lateral sclerosis (ALS), have been associated with repeat associated non-ATG (RAN) proteins, including, for example, poly(Proline-Arginine) [poly(PR)], poly(Glycine-Proline) [poly(GP)], and poly(Glycine-Alanine) [poly(GA)], and poly(Glycine-Arginine) [poly(GR)], etc. In these diseases, expansion mutations have been shown to undergo a novel type of protein translation that occurs in multiple reading frames and does not require a canonical AUG initiation codon. This type of translation is called repeat associated non-ATG (RAN) translation and the proteins that are produced are called RAN proteins. There is growing evidence that RAN proteins are toxic and contribute to the etiology of diseases in which they are expressed, such as ALS. It therefore is important to develop therapeutic strategies that reduce the level of RAN proteins to treat neurological diseases caused by repeat expansion mutations.

Expansion of a GGGGCC hexanucleotide sequence within the intron of the human C9orf72 gene is the most commonly known genetic cause of ALS and frontotemporal dementia (FTD) in humans. ALS may present as either familial ALS or sporadic ALS (sALS), with sALS occurring in over 90% of ALS patients. Within the sALS population, patients may be positive or negative for the C9orf72 expansion mutation. While RAN proteins have been observed in C9orf72 positive (C9+) sALS (see, e.g., Prudencio, et al. (2015), Nat Neurosci, 18 (8)), the presence of RAN proteins in C9orf72 negative (C9−) sALS was heretofore unknown and unreported in the art.

The disclosure is based, in part, on the surprising discovery that certain RAN proteins, including, for example, poly(PR), poly(GP), poly(GA), and poly(GR), are expressed and/or accumulate in the brains of certain subjects having a genetically unknown form sALS, and that these RAN proteins can be detected in a biological sample (e.g., blood, serum, or cerebrospinal fluid (CSF)) obtained from the subject. By “genetically unknown,” it is meant that (1) the subjects do not comprise an expansion mutation within a C9orf72 and/or an SCA36 genetic locus, and therefore that the observed RAN proteins are not expressed from a C9orf72 and/or an SCA36 genetic locus, and (2) the gene or genes expressing the observed RAN proteins are unknown and unidentified. Notably, the methods and compositions of the disclosure identify certain gene or genes which comprise mutation(s) leading to the expression of the observed RAN proteins, and which were previously unknown to be associated with sALS. The methods described herein therefore represent an unexpected and surprising advancement over the art, because said gene(s) can be used to identify subjects having, suspected of having, or at risk of developing sALS which is unrelated to expansion mutations within the C9orf72 and/or SCA36 genetic loci (e.g., C9− sALS).

Aspects of the disclosure relate to a method comprising: (i) obtaining a biological sample from a subject; (ii) detecting in the biological sample obtained from the subject at least one RAN protein; and (iii) when at least one RAN protein is detected in (ii), determining that the at least one RAN protein is not expressed from a C9orf72 or SCA36 locus of the subject. In some embodiments, the at least one RAN protein is expressed from an ADAMTS14 locus of the subject.

Aspects of the disclosure relate to a method comprising: (i) detecting in a biological sample obtained from a subject at least one RAN protein; (ii) when at least one RAN protein is detected in (i), determining that the at least one RAN protein is not expressed from a C9orf72 or SCA36 locus of the subject; and (iii) identifying the subject has having or being at risk of developing sporadic ALS (sALS) based on the detecting of at least one RAN protein not expressed from a C9orf72 or SCA36 locus of the subject.

Aspects of the disclosure relate to a method for diagnosing C9− sALS. In some embodiments, the method comprises: (i) detecting in a biological sample obtained from a subject at least one RAN protein; and (ii) diagnosing the subject as having C9− sALS based upon the presence of the at least one RAN protein. In some embodiments, the at least one RAN protein is not expressed from a C9orf72 or SCA36 locus of the subject. In some embodiments, accumulations of repeat containing sense or antisense RNA containing repeat expansions may be detected using fluorescence in situ hybridization (FISH) probes to detect the accumulating RNA. In some embodiments, the RNA accumulations present in subjects having or at risk of developing C9− sALS are not expressed from a C9orf72 or SCA36 locus in the subject.

In some embodiments, the method comprises: (i) detecting in a biological sample obtained from a subject at least one RAN protein; (ii) when at least one RAN protein is detected in (i), determining that the at least one RAN protein is not expressed from a C9orf72 locus of the subject; and (iii) diagnosing the subject as having C9− sALS based on the presence of the at least one RAN protein that was not expressed from the C9orf72 locus.

In some embodiments, the step of detecting comprises performing an assay on the biological sample.

In some embodiments, the any of the methods of the present disclosure further comprise a step of administering to the identified or diagnosed subject a therapeutic agent for the treatment of the sALS.

Aspects of the disclosure relate to a method for treating C9− sALS in a subject. In some embodiments, the method comprises administering to the subject a therapeutic agent for the treatment of C9− sALS. In some embodiments, the subject has been diagnosed as having C9− sALS according to any of the methods described herein.

In some embodiments, the biological sample is blood, serum, or cerebrospinal fluid (CSF). In some embodiments, the subject is a mammalian subject, optionally a human subject or a mouse subject.

In some embodiments, 1, 2, 3, or 4 RAN proteins are detected. In some embodiments, the at least one RAN protein is a poly(GP), poly(GA), poly(GR), and/or poly(PR) RAN protein.

In some embodiments, the at least one RAN protein is encoded by a gene comprising between 2 and 10,000 repeats of a nucleic acid sequence as set forth in any of Tables 1-4. In some embodiments, the at least one RAN protein is encoded by a gene selected from Table 6. In some embodiments, the at least one RAN protein is encoded by Rab20 or ADAMTS14. In some embodiments, an assay is used to determine whether a RAN protein was expressed from one or more genes selected from Table 6. In some embodiments, the assay for identifying RNA foci is Fluorescence In situ Hybridization (FISH), probes=fluorophore (e.g., Cy3, Cy5, A555, A549, A488)-labeled DNA sequences that complement to repeat sequences at expanded loci. In some embodiments, the assay for identifying RNA foci is deactivated Cas9-based hybridization: probes=DNA sequences that complement to repeat sequences at expanded loci and fluorophore-labeled deactivated Cas9 (dCas9).

In some embodiments, the number of poly amino acid repeats in the at least one RAN protein is at least 40.

In some embodiments, an antigen retrieval method is performed on the biological sample prior to the detecting.

In some embodiments, the therapeutic agent comprises a small molecule, interfering nucleic acid, DNA aptamer, RNA aptamer, protein, or antibody.

In some embodiments, the small molecule comprises an inhibitor of eukaryotic initiation factor 2 (eIF2), eukaryotic initiation factor 3 (eIF3), protein kinase R (PKR), p62, LC3 I subunit, LC3 II subunit, TARBP2, or Toll-like receptor 3 (TLR3). In some embodiments, the small molecule comprises metformin or a pharmaceutically acceptable salt, co-crystal, tautomer, stereoisomer, solvate, hydrate, polymorph, isotopically enriched derivative, or prodrug thereof; buformin; or phenformin.

In some embodiments, the interfering nucleic acid comprises a dsRNA, siRNA, shRNA, miRNA, artificial miRNA (ami-RNA), or antisense oligonucleotide (ASO). In some embodiments, the interfering nucleic acid inhibits expression of eukaryotic initiation factor 2 (eIF2), eukaryotic initiation factor 3 (eIF3), protein kinase R (PKR), p62, LC3 I subunit, LC3 II subunit, Toll-like receptor 3 (TLR3), a gene comprising a nucleic acid sequence as set forth in any one of Tables 1-4, or a gene selected from Table 6. In some embodiments, the interfering nucleic acid inhibits expression of one or more eIF3 subunits selected from the group consisting of eIF3a, eIF3b, eIF3c, eIF3d, eIF3e, eIF3f, eIF3g, eIF3h, eIF3i, eIF3j, eIF3k, eIF3l, and eIF3m. In some embodiments, the interfering nucleic acid comprises a region of complementarity with any one of the nucleic acid sequences set forth in Tables 1-4.

In some embodiments, the protein inhibits eIF2, eIF3, PKR, p62, LC3 I subunit, LC3 II subunit, TLR3, a gene comprising a nucleic acid sequence set as forth in any one of Tables 1-4, or a gene selected from Table 6. In some embodiments, the protein is a dominant-negative variant of PKR. In some embodiments, the dominant-negative variant comprises a mutation at amino acid position 296, optionally wherein the mutation is K296R. In some embodiments, the protein is delivered to the subject by a vector. In some embodiments, the vector is a viral vector, optionally a recombinant adeno-associated virus (rAAV). In some embodiments, the rAAV comprises an AAV9 capsid protein or variant thereof.

In some embodiments, the antibody targets eIF2, eIF3, PKR, p62, LC3 I subunit, LC3 II subunit, TLR3, or one or more RAN proteins. In some embodiments, the one or more RAN proteins is a poly(GR), poly(GP), poly(PR), and/or poly(GA) RAN protein(s). In some embodiments, the antibody specifically binds to the poly-amino acid repeat of the one or more RAN protein(s). In some embodiments, the antibody specifically binds to the C-terminus of the one or more RAN protein(s). In some embodiments, the antibody is a monoclonal antibody or a polyclonal antibody.

In some embodiments, the therapeutic agent inhibits translation of one or more RAN proteins.

In some embodiments, the methods of the present disclosure further comprise administering a second therapeutic agent to the subject. In some embodiments, the second therapeutic agent is a therapeutic agent approved by the FDA for treatment of ALS, including sALS. In some embodiments, the second therapeutic agent is selected from donepezil, galantamine, memantine, rivastigimine, or a combination thereof.

In some embodiments, detection of the one or more RAN proteins comprises performing a binding assay (e.g., an antibody-based binding assay), electrochemiluminescence-based immunoassay (e.g., Meso Scale Discovery (MSD) immunoassay), hybridization assay, immunoblot analysis, Western blot analysis, immunohistochemistry, dot blot assay, and/or enzyme-linked immunosorbent assay (ELISA) (e.g., tolling circle amplification (RCA)-based ELISA, real-time polymerase chain reaction (rtPCR)-based ELISA, digital ELISA such as single molecule array (SIMOA), etc.). In some embodiments, a hybridization assay comprises contacting a sample with one or more detectable nucleic acid probes (e.g., detectable nucleic acid probes that specifically bind to sequences encoding RAN proteins). In some embodiments, the assay comprises antibodies against repeat motifs of RAN proteins described herein. In some embodiments, the assay comprises antibodies against C-terminal specific sequences of RAN proteins. In some embodiments, a hybridization assay comprises Fluorescence In situ Hybridization (FISH) and/or dCas9-based enrichment. In some embodiments, an assay is used for detecting expansion mutations. In some embodiments, the assay for detecting expansion mutations is repeat prime PCR, long-range PCR, and/or Southern blot. These assays use primers that bind to DNA sequences within and upstream, and/or downstream of the repeat or flanking the repeat.

In some embodiments, the detecting is performed by dot blot, binding assay, hybridization assay, immunoblot analysis, 2-D gel electrophoresis, Western blot, immunohistochemistry (IHC), ELISA, RCA-based ELISA, rtPCR-based ELISA, label free immunoassays such as surface plasmon resonance bio layer interferometry, immunoquantitative PCR, mass spectrometry such as GC-MS, LC-MS, MALDI-TOF-MS, bead based immunoassays, immunoprecipitation, immunostaining, or immunoelectrophoresis.

In some embodiments, the ELISA is RCA-based ELISA or rtPCR-based ELISA.

In some embodiments, the Western blot comprises contacting the sample with an anti-RAN antibody. In some embodiments, the anti-RAN antibody targets a poly(GP), poly(GR), poly(PR), and/or poly(GA) repeat region of a RAN protein. In some embodiments, the anti-RAN antibody targets the C-terminus of a RAN protein that comprises an amino acid sequence that is not the repeat amino acid sequences poly(GP), poly(GA), poly(GR), or poly(PR).

In some embodiments, the hybridization assay comprises Fluorescence In situ Hybridization (FISH).

In some embodiments, the hybridization assay comprises dCas9-based enrichment. In some embodiments, dCas9-based enrichment is performed using a Streptococcus pyogenes-derived dCas9 (spdCas9) molecule. In some embodiments, the dCas9-based enrichment is performed using a Cas9 protein that is a mutant of a wild-type Cas9. In some embodiments, the dCas9-based enrichment is performed using a Cas9 protein that comprises a mutation that inactivates a Cas9 nuclease activity. In some embodiments, the mutation comprises a mutation in a DNA-cleavage domain of a Cas9 molecule. In some embodiments, the mutation comprises a mutation in a RuvC domain and/or a mutation in a HNH domain. In some embodiments, the dCas9 protein comprises a Staphylococcus aureus dCas9, a Streptococcus pyogenes dCas9, a Campylobacter jejuni dCas9, a Corynebacterium diphtheria dCas9, a Eubacterium ventriosum dCas9, a Streptococcus pasteurianus dCas9, a Lactobacillus farciminis dCas9, a Sphaerochaeta globus dCas9, an Azospirillum (e.g., strain B510) dCas9, a Gluconacetobacter diazotrophicus dCas9, a Neisseria cinerea dCas9, a Roseburia intestinalis dCas9, a Parvibaculum lavamentivorans dCas9, a Nitratifractor salsuginis (e.g., strain DSM 16511) dCas9, a Campylobacter lari (e.g., strain CF89-12) dCas9, or a Streptococcus thermophilus (e.g., strain LMD-9) dCas9.

In some embodiments, the detecting comprises contacting the sample with an anti-RAN antibody. In some embodiments, the anti-RAN protein antibody targets a poly(GP), poly(GR), poly(PR), and/or poly(GA) repeat region of a RAN protein. In some embodiments, the anti-RAN protein antibody targets the C-terminus of a RAN protein that comprises an amino acid sequence that is not the repeat amino acid sequences poly(GP), poly(GA), poly(GR), or poly(PR).

In some embodiments, the detecting further comprises nucleic acid sequencing. In some embodiments, the nucleic acid sequencing is Next-Generation Sequencing (NGS). In some embodiments, the step of nucleic acid sequencing is performed either with or without also performing an enrichment step on the sample. In some embodiments, the enrichment step comprises dCas9-based enrichment. In some embodiments, the dCas9-based enrichment uses guideRNAs. In some embodiments, the guideRNAs used in the dCas9-based enrichment target NGG protospacer adjacent motifs (PAM) containing repeats. In some embodiments, the guideRNAs used in the dCas9-based enrichment target non-NGG PAM containing repeats. In some embodiments, the non-NGG PAM containing repeats comprise CAG and CTG expansion repeats. In some embodiments, the guideRNAs used in the dCas9-based enrichment enriches non-NGG PAM containing repeat expansions that are longer (e.g., 1, 2, 3, 4, 5, 6, 7, 8, 9, 10, 11, 12, 13, 14, 15, 20, 25, 30, 35, 40, 45, 50, 55, 60, 70, 80, 90, 100 repeats longer) than the corresponding normal allele. In some embodiments, the guideRNAs used in the dCas9-based enrichment identify multiple repeat expansions simultaneously, including, in some embodiments, sequences with non-NGG PAMs.

Aspects of the disclosure relate to a method of monitoring a C9− sALS therapeutic regimen. In some embodiments, the method comprises: (i) detecting in a second biological sample obtained from a subject that has been administered a therapeutic regimen for C9− sALS a level of one or more poly(GR), poly(GP), poly(GA), and/or poly(PR) repeat-associated non-ATG (RAN) protein(s); (ii) comparing the level of one or more RAN proteins detected in (i) to a level of the same RAN protein(s) in a first biological sample obtained from the subject prior to being administered the therapeutic regimen; and (iii) continuing to administer the therapeutic regimen for C9− sALS when the level of the one or more RAN protein(s) in the second biological sample is reduced compared to the level of the first biological sample. In some embodiments, the first biological sample and/or the second biological sample is blood, serum or cerebrospinal fluid (CSF).

Aspects of the disclosure relate to therapeutic agents for the treatment of C9− sALS, wherein the therapeutic agent is a small molecule, interfering nucleic acid, DNA aptamer, RNA aptamer, protein, or antibody that reduces expression of one or more poly(GR), poly(GP), poly(GA), and/or poly(PR) RAN protein(s).

### BRIEF DESCRIPTION OF DRAWINGS

FIG. 1 shows an example of a workflow according to embodiments of the present disclosure.

FIGS. 2A-2B show representative data of GR and PR aggregates in genetically unknown sporadic ALS autopsy (sALS) brains. FIG. 2A shows GR aggregates. FIG. 2B shows PR aggregates.

FIG. 3 shows a schematic depicting dCas9 repeat expansion enrichment.

FIG. 4 shows dCas9-based repeat expansion enrichment and detection (dCas9READ). Favored binding of sgRNA-dCas9 complexes to repeat expansion allows the enrichment and the identification of the repeat and unique flanking sequence. Repeat expansions were pulled-down, and repeat expansion and their unique flanking sequences were identified using next generation sequencing (NGS).

FIGS. 5A-5B show an example of how the dCas9READ method described herein can be used to isolate a GGGGCC (G4C2) repeat expansion motif. While the G4C2 repeat expansion motif is associated with cases of C9+ ALS, it can also be associated with C9-ALS. FIG. 5A shows validation of the dCas9READ enrichment method using quantitative polymerase chain reaction (qPCR). FIG. 5B shows next generation sequencing (NGS) mapping of the C9 locus.

FIG. 6 shows representative data demonstrating that the dCas9READ method described herein can be used with a mixture of sgRNAs, and that multiple repeats can be screened simultaneously using dCas9READ. sgRNAs can be developed using the nucleic acid sequences encoding each type of di-peptide repeat motif (e.g., as shown in Tables 1-4).

FIG. 7 shows an example of a pathology-to-genetics strategy for studying novel repeat expansion mutations, as described herein.

FIG. 8 shows representative immunohistochemistry (IHC) data indicating that poly(PR) RAN proteins were present in 22% of sALS brain tissue samples tested.

FIG. 9 shows representative data indicating poly(PR) sALS samples are negative for C9orf72 and SCA36 expansion repeats, and that poly(PR) RAN proteins found in sALS samples are expressed from novel repeat expansions.

FIG. 10 shows representative data indicating poly(PR) RAN protein aggregates were detected in sALS brain organoids generated from patient-derived iPSCs generated from blood samples.

FIGS. 11A-11C show schematics indicating two repeat expansion configurations were identified between exons 2 and 3 of ADAMTS14. FIG. 11A shows a schematic of the insertion and deletion for each configuration. FIG. 11B shows a schematic for poly(GR) RAN translation from an ADAMTS14 allele. FIG. 11C shows a schematic for poly(PR) RAN translation from an ADAMTS14 allele.

FIG. 12 shows representative gel images of sALS, C9orf72 ALS, and control samples with or without ADAMTS14 poly(GR)-encoding repeat expansions.

### DETAILED DESCRIPTION

In some aspects, the disclosure relates to methods and compositions that are useful for detecting RAN proteins in biological samples which do not comprise an expansion mutation in a C9orf72 and/or SCA36 locus. In some embodiments, the methods and compositions of the disclosure identify certain gene or genes which comprise mutation(s) leading to the expression of the detected RAN proteins, and which were previously unknown to be associated with sALS. In some embodiments, said gene(s) can be used to identify or diagnose subjects having, suspected of having, or at risk of developing sALS which is unrelated to expansion mutations within the C9orf72 and/or SCA36 genetic loci (e.g., C9− sALS). In some embodiments, subjects identified or diagnosed according to the methods of the present disclosure are administered a therapeutic agent for the treatment of sALS.

Aspects of the disclosure relate to certain repeat-associated non-ATG (RAN) proteins (e.g., poly(PR), poly(GR); poly(GP); and poly(GA)) which are expressed from a genetic locus of a subject that is not C9orf72 or SCA36, and are detectable in biological samples of subjects having or suspected of having C9− sALS. Biological samples can be any specimen derived or obtained from a subject having or suspected of having C9− sALS. In some embodiments, the biological sample is blood, serum (e.g., plasma from which the clotting proteins have been removed) or cerebrospinal fluid (CSF). In some embodiments, a biological sample is a tissue sample, for example central nervous system (CNS) tissue, such as brain tissue or spinal cord tissue. The skilled artisan will recognize other biological samples, such as cells (e.g., brain cells, neuronal cells, skin cells, etc.) suitable for methods described by the disclosure.

A “subject having or suspected of having C9− sALS” generally refers to a subject (1) exhibiting one or more signs and symptoms of sALS, including but not limited to: difficulty walking or doing normal daily activities; tripping and falling; weakness in legs, feet or ankles; hand weakness or clumsiness; slurred speech or trouble swallowing; muscle cramps and twitching in arms, shoulders and tongue; inappropriate crying, laughing or yawning; and/or cognitive and behavioral changes, and (2) who does not comprise an expansion mutation in the C9orf72 and/or SCA36 gene. A subject can be a mammal (e.g., human, mouse, rat, dog, cat, or pig). In some embodiments, a subject is a non-human animal, for example a mouse, rat, guinea pig, cat dog, horse, camel, etc. In some embodiments, the subject is a human.

A subject “at risk of developing C9− sALS” is one who does not exhibit one or more signs or symptoms of sALS, but who comprises an expansion mutation in one or more gene(s) set forth in Table 6. In some embodiments, a subject who is at risk of developing C9− sALS has a higher risk of developing C9− sALS than subject who does not comprise an expansion mutation in one or more genes set forth in Table 6. In some embodiments, a subject who is at risk of developing C9− sALS has at least a 5%, 10%, 15%, 20%, 25%, 30%, 35%, 40%, 45%, 50%, 55%, 60%, 65%, 70%, 75%, 80%, 85%, 90%, 95%, or 99% higher risk of developing C9− sALS than subject who does not comprise an expansion mutation in one or more genes set forth in Table 6.

### RAN Proteins

A “RAN protein (repeat-associated non-ATG translated protein)” is a polypeptide that is translated from sense or antisense RNA sequences bidirectionally expressed from a repeat expansion mutation in the absence of an AUG initiation codon. RAN protein-encoding sequences can be found in the genome at multiple loci.

Generally, RAN proteins comprise expansion repeats of a single amino acid, di-amino acid, tri-amino acid, or quad-amino acid (e.g., tetra-amino acid) repeat units, termed “poly amino acid repeats.” Examples of di-amino acid RAN proteins include GPGPGPGPGP (poly-GP) (SEQ ID NO: 1), GAGAGAGAGA (poly-GA) (SEQ ID NO: 2), GRGRGRGRGR (poly-GR) (SEQ ID NO: 3), and PRPRPRPRPR (poly-PR) (SEQ ID NO: 4). RAN proteins can have a poly amino acid repeat of at least 20, at least 25, at least 30, at least 40, at least 50, at least 60, at least 70, at least 80, at least 90, at least 100, or at least 200 amino acid residues in length. In some embodiments, a RAN protein has a poly amino acid repeat more than 200 amino acid residues (e.g., 500, 1000, 5000, 10,000, etc.) in length. In some embodiments, the number of poly amino acid repeats in the at least one RAN protein is at least 40.

Generally, RAN proteins are translated from abnormal repeat expansions (e.g., TCT repeats, hexanucleotide repeats, etc.) of DNA. The disclosure is based, in part, on the identification of microsatellite repeats in certain subjects having C9− sALS characterized by expression of one or more (e.g., 2, 3, 4, 5, or more) RAN proteins, for example poly(PR); poly(GR); poly(GP); and/or poly(GA). In some embodiments, the disease status of a subject having or suspected of having C9− sALS is classified by the number and/or type of microsatellite repeats present (e.g., detected) in the subject (e.g., in the genome of a subject or in a gene of the subject). In some embodiments, a subject having less than 10 repeat sequences does not exhibit signs or symptoms of C9− sALS. In some embodiments, a subject having between 10 and 40 repeats may or may not exhibit one or more signs or symptoms of C9− sALS. In some embodiments, a subject having more than 40 repeats exhibits one or more signs or symptoms of C9− sALS. In certain cases, a subject is identified as having C9− sALS via the characterization of a large (>100) number of repeats.

Microsatellite repeat sequences encoding RAN proteins are generally known, and examples of nucleic acid sequences encoding poly(PR), poly(GR), poly(GA), and poly(GP) RAN proteins are shown in Tables 1-4, respectively. However, it will be understood that RAN proteins may contain multiple di-amino acid repeats, as described elsewhere herein. Nucleic acid sequences encoding a RAN protein (e.g., containing multiple di-amino acid repeats) may in some embodiments comprise multiple iterations of any of the sequences shown in Tables 1-4.

In some embodiments, a subject having, suspected of having, or at risk of developing C9− sALS has one or more microsatellite repeat sequences encoding a poly(PR) RAN protein. Examples of microsatellite repeat sequences encoding poly(PR) proteins are shown in Table 1.

TABLE 1
Nucleic acid sequences encoding PR

| | | | |
|---|---|---|---|
| CCTCGT | CCCCGT | CCACGT | CCGCGT |
| CCTCGC | CCCCGC | CCACGC | CCGCGC |
| CCTCGA | CCCCGA | CCACGA | CCGCGA |
| CCTCGG | CCCCGG | CCACGG | CCGCGG |
| CCTAGA | CCCAGA | CCAAGA | CCGAGA |
| CCTAGG | CCCAGG | CCAAGG | CCGAGG |

In some embodiments, a subject having, suspected of having, or at risk of developing C9− sALS has one or more microsatellite repeat sequences encoding a poly(GR) RAN protein. Examples of microsatellite repeat sequences encoding poly(GR) proteins are shown in Table 2.

TABLE 2
Nucleic acid sequences encoding GR

| | | | |
|---|---|---|---|
| GGTCGT | GGCCGT | GGACGT | GGGCGT |
| GGTCGC | GGCCGC | GGACGC | GGGCGC |
| GGTCGA | GGCCGA | GGACGA | GGGCGA |
| GGTCGG | GGCCGG | GGACGG | GGGCGG |
| GGTAGA | GGCAGA | GGAAGA | GGGAGA |
| GGTAGG | GGCAGG | GGAAGG | GGGAGG |

In some embodiments, a subject having, suspected of having, or at risk of developing C9− sALS has one or more microsatellite repeat sequences encoding a poly(GA) RAN protein. Examples of microsatellite repeat sequences encoding poly(GA) proteins are shown in Table 3.

TABLE 3
Nucleic acid sequences encoding GA

| | | | |
|---|---|---|---|
| GGTGCT | GGCGCC | GGAGCA | GGGGCG |
| GGTGCC | GGCGCA | GGAGCG | GGGGCT |
| GGTGCA | GGCGCG | GGAGCT | GGGGCC |
| GGTGCG | GGCGCT | GGAGCC | GGGGCA |

In some embodiments, a subject having, suspected of having, or at risk of developing C9− sALS has one or more microsatellite repeat sequences encoding a poly(GP) RAN protein. Examples of microsatellite repeat sequences encoding poly(GP) proteins are shown in Table 4.

TABLE 4
Nucleic acid sequences encoding GP

| | | | |
|---|---|---|---|
| GGTCCT | GGCCCC | GGACCA | GGGCCG |
| GGTCCC | GGCCCA | GGACCG | GGGCCT |
| GGTCCA | GGCCCG | GGACCT | GGGCCC |
| GGTCCG | GGCCCT | GGACCC | GGGCCA |

In some aspects, the disclosure relates to the discovery that RAN protein (e.g., poly(PR); poly(GR); poly(GP); poly(GA)) aggregation patterns are length-dependent. For example, RAN proteins having poly amino acid repeats that are >20, >48, or >80 residues in length aggregate differently in the brain of a subject. Generally, the differential aggregation properties of RAN proteins having different lengths can be used to detect RAN proteins in a biological sample. Longer RAN proteins are found at higher levels in biological samples, such as blood, serum, or CSF. In some embodiments, RAN proteins having poly amino acid repeats >40, >50, >60, >70, or >80 amino acid residues in length are detectable in a biological sample.

In some embodiments, a subject having or suspected of having C9− sALS has one or more microsatellite repeat sequences encoding a poly(PR), poly(GR), poly(GA), or poly(GP) RAN protein, wherein the microsatellite repeat sequences are comprised within a gene selected from Table 6. In some embodiments, a subject having or suspected of having C9− sALS has one or more microsatellite repeat sequences encoding a poly(PR), poly(GR), poly(GA), or poly(GP) RAN protein, wherein the microsatellite repeat sequences are comprised within a Rab20 gene.

### Methods of Detecting RAN Proteins

The disclosure is based, in part, on the discovery that certain biological sample processing methods (e.g., antibody-based capture, hybridization-based assays, dCas9-based enrichment, or combinations thereof) enable the reproducible detection of one or more RAN proteins in a biological sample. In some embodiments of methods described by the disclosure, a sample (e.g., a biological sample) is treated by an antibody-based capture process to isolate one or more RAN proteins within the sample. Typically, the antibody-based capture methods include contacting the sample with one or more (e.g., 2, 3, 4, 5, or more) anti-RAN protein antibodies. In some embodiments, the one or more anti-RAN antibodies are conjugated to a solid support (e.g., a scaffold, resin beads, etc.). In some embodiments, antibody-based capture methods comprise physically separating and/or isolating RAN proteins that have been bound by the anti-RAN antibody(s), for example eluting the RAN proteins by a chromatographic method such as affinity chromatography or ion-exchange chromatography.

A biological sample may be subjected to an antigen retrieval procedure prior to being contacted with an anti-RAN antibody. As used herein, “antigen retrieval” (also referred to as epitope retrieval, or antigen unmasking) refers to a process in which a biological sample (e.g., blood, serum, CSF, etc.) are treated under conditions which expose antigens (e.g., epitopes) that were previously inaccessible to detection agents (e.g., antibodies, aptamers, and other binding molecules) prior to the process. Generally, antigen retrieval methods comprise steps including but not limited to heating, pressure treatment, enzymatic digestion, treatment with reducing agents, treatment with oxidizing agents, treatment with crosslinking agents, treatment with denaturing agents (e.g., detergents, ethanol, acids), or changes in pH, or any combination of the foregoing. Several antigen retrieval methods are known in the art, including but not limited to protease-induced epitope retrieval (PIER) and heat-induced epitope retrieval (HIER). In some embodiments, antigen retrieval procedures reduce the background and increase the sensitivity of detection techniques (e.g., immunohistochemistry (IHC), immuno-blot (such as Western Blot), ELISA, etc.).

Detection of RAN proteins in a biological sample may be performed by Western blot. Western blots generally employ the use of a detection agent or probe to identify the presence of a protein or peptide. In some embodiments, detection of one or more RAN proteins is performed by immunoblot (e.g., dot blot, 2-D gel electrophoresis, Western Blot, etc.), immunohistochemistry (IHC), ELISA (e.g., RCA-based ELISA or rtPCR-based ELISA), label free immunoassays such as surface plasmon resonance bio layer interferometry, immunoquantitative PCR, mass spectrometry such as GC-MS, LC-MS, MALDI-TOF-MS, bead-based immunoassays, immunoprecipitation, immunostaining, or immunoelectrophoresis. In some embodiments, the detection agent is an antibody. In some embodiments, the antibody is an anti-RAN protein antibody, such as anti-poly(GR), anti-poly(PR), anti-poly(GA), or anti-poly(GP). In some embodiments, an anti-RAN protein antibody targets (e.g., specifically binds to) the amino acid repeat region (e.g., PRPRPRPRPR (SEQ ID NO: 4), GRGRGRGRGR (SEQ ID NO: 3), GAGAGAGAGA (SEQ ID NO: 2), GPGPGPGPGP (SEQ ID NO: 1), etc.) of a RAN protein. In some embodiments, an anti-RAN protein antibody targets (e.g., specifically binds to) an epitope comprising amino acids in the characteristic reading frame specific C-terminus translated 3′ of the repeated amino acids. In some embodiments, an anti-RAN protein antibody targets (e.g., specifically binds to) an epitope comprising amino acids bridging the C terminus of the amino acid repeat region and the N terminus of the characteristic reading-frame specific C-terminus translated 3′ of the repeated amino acids.

In some embodiments, an anti-RAN antibody targets (e.g., specifically binds to) any portion of a RAN protein that does not comprise the poly amino acid repeat, for example the C-terminus of a RAN protein (e.g., the C-terminus of a poly(GR), poly(PR), poly(GP), or poly(GA) RAN protein). Examples of anti-RAN antibodies targeting RAN protein poly amino acid repeats are disclosed, for example, in International Application Publication No. WO 2014/159247, the entire content of which is incorporated herein by reference. Examples of anti-RAN antibodies targeting the C-terminus of RAN protein are disclosed, for example, in U.S. Publication No. 2013/0115603, the entire content of which is incorporated herein by reference. In some embodiments, a set (or combination) of anti-RAN antibodies (e.g., a combination of two or more anti-RAN antibodies selected from anti-poly(GR), anti-poly(PR), anti-poly(GP), anti-poly(G), and/or anti-poly(GA)), is used to detect one or more RAN proteins in a biological sample.

An anti-RAN antibody can be a polyclonal antibody or a monoclonal antibody. Typically, polyclonal antibodies are produced by inoculation of a suitable mammal, such as a mouse, rabbit or goat. Larger mammals are often preferred as the amount of serum that can be collected is greater. An antigen is injected into the mammal. This induces the B-lymphocytes to produce IgG immunoglobulins specific for the antigen. This polyclonal IgG is purified from the mammal's serum. Monoclonal antibodies are generally produced by a single cell line (e.g., a hybridoma cell line). In some embodiments, an anti-RAN antibody is purified (e.g., isolated from serum). In some embodiments, the antigen is 12-20 amino acids. For antibodies against repeat motifs, an antigen is a repeat sequence. For antibodies against C-terminal sequence of a RAN protein, an antigen is a C-terminal specific sequence. In some embodiments, an antigen is a portion of a C-terminal sequence, for example, a fragment of the C-terminal sequences that is 3-5 or 5-10, or more amino acids in length, for example, 6, 7, 8, 9, 10, 11, 12, 13, 14, 15, 16, 17, 18, 19, 20, 25, 30, 35, 40, or 50 amino acids in length (e.g., from one of the C-terminal sequences described in this application).

In some embodiments, the disclosure provides methods of producing an antibody, the method comprising administering to the subject a peptide antigen comprising a RAN protein repeat sequence, for example anti-poly(GR), anti-poly(PR), anti-poly(GP), anti-poly(G), and/or anti-poly(GA). In some embodiments, the subject is a mammal, for example a non-human primate, rodent (e.g., rat, hamster, guinea pig, etc.). In some embodiments, the subject is a human (e.g., a subject is injected with a peptide antigen for the purposes of eliciting a host antibody response against the peptide antigen, for example a RAN protein). In some embodiments, an antibody is produced by expressing in a cell (e.g., a B-cell, hybridoma cell, etc.) one or more RAN proteins or RAN protein repeat sequences.

Numerous methods may be used for obtaining anti-RAN antibodies. For example, antibodies can be produced using recombinant DNA methods. Monoclonal antibodies may also be produced by generation of hybridomas (see, e.g., Kohler and Milstein (1975) Nature, 256:495-499) in accordance with known methods. Hybridomas formed in this manner are then screened using standard methods, such as enzyme-linked immunosorbent assay (ELISA; e.g., RCA-based ELISA or rtPCR-based ELISA) and surface plasmon resonance (e.g., OCTET or BIACORE) analysis, to identify one or more hybridomas that produce an antibody that specifically binds with a specified antigen. Any form of the specified antigen (e.g., a RAN protein) may be used as the immunogen, e.g., recombinant antigen, naturally occurring forms, any variants or fragments thereof. One exemplary method of making antibodies includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228:1315-1317; Clackson et al. (1991) Nature, 352:624-628; Marks et al. (1991) J. Mol. Biol., 222:581-597WO92/18619; WO 91/17271; WO 92/20791; WO 92/15679; WO 93/01288; WO 92/01047; WO 92/09690; and WO 90/02809.

In addition to the use of display libraries, the specified antigen (e.g., one or more RAN proteins) can be used to immunize a non-human animal, e.g., a rodent, e.g., a mouse, hamster, or rat. In one embodiment, the non-human animal is a mouse.

In another embodiment, a monoclonal antibody is obtained from the non-human animal, and then modified, e.g., made chimeric, using recombinant DNA techniques known in the art. A variety of approaches for making chimeric antibodies have been described. See, e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B.

Antibodies can also be humanized by methods known in the art. For example, monoclonal antibodies with a desired binding specificity can be commercially humanized (Scotgene, Scotland; and Oxford Molecular, Palo Alto, Calif.). Fully humanized antibodies, such as those expressed in transgenic animals are within the scope of the invention (see, e.g., Green et al. (1994) Nature Genetics 7, 13; and U.S. Pat. Nos. 5,545,806 and 5,569,825).

For additional antibody production techniques, see, Antibodies: A Laboratory Manual, Second Edition. Edited by Edward A. Greenfield, Dana-Farber Cancer Institute, @2014. The present disclosure is not necessarily limited to any particular source, method of production, or other special characteristics of an antibody.

In some embodiments, methods of detecting one or more RAN proteins in a biological sample are useful for monitoring the progress of C9− sALS. For example, in some embodiments, biological samples are obtained from a subject prior to and after (e.g., 1 week, 2 weeks, 1 month, 6 months, or one year after) commencement of a therapeutic regimen and the amount of RAN proteins detected in the samples is compared. In some embodiments, if the level (e.g., amount) of RAN protein in the post-treatment sample is reduced compared to the pre-treatment level (e.g., amount) of RAN protein, the therapeutic regimen is successful. In some embodiments, the level of RAN proteins in biological samples (e.g., 1, 2, 3, 4, 5, 6, 7, 8, 9, 10, or more samples) of a subject are continuously monitored during a therapeutic regimen (e.g., measured on 1, 2, 3, 4, 5, 6, 7, 8, 9, 10, or more separate occasions).

In some embodiments, a detection agent is an aptamer (e.g., RNA aptamer, DNA aptamer, or peptide aptamer). In some embodiments, an aptamer specifically binds to a RAN protein (e.g., poly(PR), poly(GR), poly(GP), and/or poly(GA)).

Aspects of the disclosure relate to nucleic acid hybridization-based methods for identifying the presence of RAN proteins or microsatellite repeat sequences encoding RAN proteins in a biological sample (e.g., a biological sample obtained from a subject). The disclosure is based, in part, on methods for detecting nucleic acid sequences encoding RAN proteins by detectable nucleic acid probes (e.g., fluorophore-conjugated DNA probes). Generally, a “detectable nucleic acid probe” refers to a nucleic acid sequence that specifically binds to (e.g., hybridizes with) a target sequence, and comprises a detectable moiety, for example a fluorescent moiety, radioactive moiety, chemiluminescent moiety, electroluminescent moiety, biotin, peptide tag (e.g., poly-His tag, FLAG-tag, etc.), etc. In some embodiments, the detectable nucleic acid probe comprises a region of complementarity (e.g., a nucleic acid sequence that is the complement of, and capable of hybridizing to) a nucleic acid sequence encoding one or more RAN proteins. A region of complementarity may range from about 2 nucleotides in length to about 100 nucleotides in length (e.g., any number of nucleotides between 2 and 100, inclusive). In some embodiments, a nucleic acid probe comprises a region of complementarity with a sequence set forth in any one of Tables 1˜4 or a region of complementarity with a repeat sequence comprising multiple repeats of a sequence set forth in any one of Tables 1-4. In some embodiments, a detectable nucleic acid probe is a DNA probe. In some embodiments, the DNA probe is conjugated to a fluorophore.

A biological sample may also be contacted with a plurality of detectable nucleic acid probes. The number of nucleic acid probes in a plurality varies. In some embodiments, a plurality of nucleic acid probes comprises between 2 and 100 (2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100) nucleic acid probes. In some embodiments, a plurality comprises more than 100 probes. The nucleic acid probes may be the same or different sequences. In some embodiments, a plurality of detectable nucleic acid probes comprises probes which hybridize to nucleic acid sequences that encode a poly(PR) RAN protein (e.g., repeat sequences set forth in Table 1). In some embodiments, a plurality of detectable nucleic acid probes comprises probes which hybridize to nucleic acid sequences that encode a poly(GR) RAN protein (e.g., repeat sequences set forth in Table 2). In some embodiments, a plurality of detectable nucleic acid probes comprises probes which hybridize to nucleic acid sequences that encode a poly(GA) RAN protein (e.g., repeat sequences set forth in Table 3). In some embodiments, a plurality of detectable nucleic acid probes comprises probes which hybridize to nucleic acid sequences that encode a poly(GP) RAN protein (e.g., repeat sequences set forth in Table 4).

In some embodiments, detectable nucleic acid probes are useful for localization of RAN protein translation by Fluorescence In situ Hybridization (FISH).

Methods for detecting one or more RAN proteins may comprise an enrichment step. “Enrichment” refers to processes which increase the amount and/or concentration of a target nucleic acid in a sample relative to other nucleic acids in a sample. Generally, enrichment may occur by increasing the number of target nucleic acid sequences in a sample (e.g., by amplifying the target sequence, for example by polymerase chain reaction (PCR), etc.), or by decreasing the amount or concentration of non-target nucleic acid sequences in the sample (e.g., by separating or isolating the target nucleic acid sequence from non-target sequences).

In some embodiments, methods described herein comprise a step of enriching a biological sample for nucleic acid sequences (e.g., microsatellite repeat sequences) encoding RAN proteins. In some embodiments, the enrichment comprises contacting the biological sample with 1) a labeled (e.g., biotinylated) dCas9 protein, and 2) one or more single-stranded guide RNA (sgRNAs) that specifically bind to nucleic acid repeat sequences encoding RAN proteins (e.g., as shown in Tables 1-4). In some embodiments, the labeled dCas9 protein and the one or more sgRNAs are provided together as a single molecule (e.g., a dCas9-sgRNA complex). In some embodiments after the biological sample with the labeled dCas9 protein and the one or more sgRNAs, the nucleic acid sequences encoding one or more RAN proteins are isolated from the labeled dCas9 protein and the sgRNAs, for example by affinity chromatography, as described by Liu et al. (2017) Cell 170:1028-1043.

In some embodiments, the detection of the one or more RAN proteins comprises Next-Generation Sequencing (NGS). In some embodiments, an enrichment step (e.g., dCas9-based enrichment) is performed on the sample, using guideRNAs. In some embodiments, the guideRNAs used in the enrichment target NGG protospacer adjacent motifs (PAM) containing repeats. In other embodiments, the guideRNAs used in the enrichment target non-NGG PAM containing repeats. In some embodiments, the non-NGG PAM containing repeats comprise CAG and CTG expansion repeats. In some embodiments, the guideRNAs used in the enrichment enrich non-NGG PAM containing repeat expansions that are longer (e.g., 1, 2, 3, 4, 5, 6, 7, 8, 9, 10, 11, 12, 13, 14, 15, 20, 25, 30, 35, 40, 45, 50, 55, 60, 70, 80, 90, 100 repeats longer) than the corresponding normal allele. In some embodiments, the guideRNAs used in the enrichment identify multiple repeat expansions simultaneously, including, in some embodiments, sequences with non-NGG PAMs.

### Therapeutic Methods

Methods of treating aC9− sALS are also contemplated by the disclosure. In some embodiments, a subject having been diagnosed with C9− sALS by a method described by the disclosure is administered a therapeutic agent.

To “treat” a disease (e.g., C9− sALS) as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host cell, tissue or organ. A therapeutically acceptable amount of an anti-RAN protein antibody may be an amount that is capable of treating a disease, e.g., C9− sALS, by reducing expression and/or aggregation of RAN proteins and/or appearance or number of RNA foci comprising RAN protein-encoding microsatellite repeat sequences. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

A therapeutic useful for treating C9− sALS can be a small molecule, protein, peptide, nucleic acid (e.g., an interfering nucleic acid), or gene therapy vector (e.g., viral vector encoding a therapeutic protein and/or an interfering nucleic acid). Therapeutics useful for treating C9− sALS may target (e.g., reduce expression, activity, accumulation, aggregation, etc.) of a RAN protein or nucleic acid encoding a RAN protein, and/or modulate the activity of another gene or gene product (e.g., protein) that interact with one or more RAN proteins. Examples of genes and gene products that interact with one or more RAN proteins include eukaryotic initiation factor 2 (eIF2), eukaryotic initiation factor 3 (eIF3), protein kinase R (PKR), p62, LC3 I subunit, LC3 II subunit, and Toll-like receptor 3 (TLR3). In some embodiments, a therapeutic agent inhibits expression or activity of one or more of eukaryotic initiation factor 2 (eIF2), eukaryotic initiation factor 3 (eIF3), protein kinase R (PKR), p62, LC3 I subunit, LC3 II subunit, and Toll-like receptor 3 (TLR3).

In some embodiments, the therapeutic agent is a small molecule. In some embodiments, the small molecule inhibits expression or activity of one or more RAN proteins. In some embodiments, a small molecule is an inhibitor of eIF3 (or an eIF3 subunit). Examples of small molecule inhibitors of eIF3 include but are not limited to mTOR inhibitors (e.g., rapamycin, PP242), S6 kinase (S6K) inhibitors, etc.

In some embodiments, the small molecule inhibits expression or activity of eukaryotic initiation factor 2A (eIF2A) or eIF2α. Examples of small molecule inhibitors of eIF2A include but are not limited to salubrinal, Sal003, ISRIB, etc. In some embodiments, the small molecule in an inhibitor of TARBP2. Examples of TARBP2 inhibitors include anti-TARBP2 antibodies, interfering RNAs (e.g., dsRNA, siRNA, shRNA, miRNA, etc.) that target anti-TARBP2, peptide inhibitors of TARBP2, and small molecule inhibitors of TARBP2. In some embodiments, the small molecule is metformin, also known as N,N-dimethylbiguanide (IUPAC N,N-Dimethylimidodicarbonimidic diamide and CAS 657-24-9), or an alternate bioactive biguanide including chloroguanide [1-[amino-(4-chloroanilino)methylidene]-2-propan-2-yl-guanidine, CAS 500-92-5], Chlorproguanil [1-[Amino-(3,4-dichloroanilino)methylidene]-2-propan-2-ylguanidine, CAS 537-21-3], buformin [N-Butylimidodicarbonimidic diamide, CAS 692-13-7] or Phenformin [2-(N-phenethylcarbamimidoyl) guanidine, CAS 114-86-3] or a pharmaceutically acceptable salt, co-crystal, tautomer, stereoisomer, solvate, hydrate, polymorph, isotopically enriched derivative, or prodrug of any of the biguanides.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. Metformin may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·xH₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred. In some embodiments, the small molecule is buformin, or phenformin.

The therapeutic agent may be an anti-RAN protein antibody. In some embodiments, the anti-RAN protein antibody is an anti-poly(GR), anti-poly(PR), anti-poly(GP), or anti-poly(GA) antibody (also referred to as α-poly(PR), α-poly(GR), etc.). An anti-RAN protein antibody may bind to an extracellular RAN protein, an intracellular RAN protein, or both extracellular and intracellular RAN proteins. In some embodiments, an anti-RAN protein antibody targets (e.g., specifically binds to) the amino acid repeat region (e.g., PRPRPRPRPR (SEQ ID NO: 4), GRGRGRGRGR (SEQ ID NO: 3), GAGAGAGAGA (SEQ ID NO: 2), GPGPGPGPGP (SEQ ID NO: 1), etc.) of a RAN protein. Examples of anti-RAN antibodies targeting RAN protein poly amino acid repeats are disclosed, for example, in International Application Publication No. WO 2014/159247, the entire content of which is incorporated herein by reference. In some embodiments, an anti-RAN protein antibody targets (e.g., specifically binds to) the amino acid repeat region of one or more RAN proteins selected from: poly(PR); poly(GR); poly(GP); and poly(GA). In some embodiments, an anti-RAN antibody targets (e.g., specifically binds to) any portion of a RAN protein that does not comprise the poly amino acid repeat, for example the C-terminus of a RAN protein (e.g., the C-terminus of a poly(GP), poly(GA), poly(GR), or poly(PR) RAN protein). Examples of anti-RAN antibodies targeting the C-terminus of RAN protein are disclosed, for example, in U.S. Publication No. 2013/0115603, the entire content of which is incorporated herein by reference. In some embodiments, a set (or combination) of anti-RAN antibodies (e.g., a combination of two or more anti-RAN antibodies selected from anti-poly(GP), anti-poly(GA), anti-poly(GR), and anti-poly(PR)) are administered to a subject for the purpose of treating a disease associated with RAN proteins.

An anti-RAN antibody can be a polyclonal antibody or a monoclonal antibody. Typically, polyclonal antibodies are produced by inoculation of a suitable mammal, such as a mouse, rabbit or goat. Larger mammals are often preferred as the amount of serum that can be collected is greater. An antigen is injected into the mammal. This induces the B-lymphocytes to produce IgG immunoglobulins specific for the antigen. This polyclonal IgG is purified from the mammal's serum. Monoclonal antibodies are generally produced by a single cell line (e.g., a hybridoma cell line). In some embodiments, an anti-RAN antibody is purified (e.g., isolated from serum).

Numerous methods may be used for obtaining anti-RAN antibodies. For example, antibodies can be produced using recombinant DNA methods. Monoclonal antibodies may also be produced by generation of hybridomas (see, e.g., Kohler and Milstein (1975) Nature, 256:495-499) in accordance with known methods. Hybridomas formed in this manner are then screened using standard methods, such as enzyme-linked immunosorbent assay (ELISA; e.g., RCA-based ELISA or rtPCR-based ELISA) and surface plasmon resonance (e.g., OCTET or BIACORE) analysis, to identify one or more hybridomas that produce an antibody that specifically binds with a specified antigen. Any form of the specified antigen (e.g., a RAN protein) may be used as the immunogen, e.g., recombinant antigen, naturally occurring forms, any variants or fragments thereof. One exemplary method of making antibodies includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228:1315-1317; Clackson et al. (1991) Nature, 352:624-628; Marks et al. (1991) J. Mol. Biol., 222:581-597WO92/18619; WO 91/17271; WO 92/20791; WO 92/15679; WO 93/01288; WO 92/01047; WO 92/09690; and WO 90/02809.

In another embodiment, a monoclonal antibody is obtained from the non-human animal, and then modified, e.g., made chimeric, using recombinant DNA techniques known in the art. A variety of approaches for making chimeric antibodies have been described. See, e.g., Morrison et al., Proc. Natl. Acad. Sci. U.S.A. 81:6851, 1985; Takeda et al., Nature 314:452, 1985, Cabilly at al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B.

Antibodies can also be humanized by methods known in the art. For example, monoclonal antibodies with a desired binding specificity can be commercially humanized (Scotgene, Scotland; and Oxford Molecular, Palo Alto, Calif.). Fully humanized antibodies, such as those expressed in transgenic animals are within the scope of the invention (see, e.g., Green et al. (1994) Nature Genetics 7, 13; and U.S. Pat. Nos. 5,545,806 and 5,569,825). For additional antibody production techniques, see, Antibodies: A Laboratory Manual, Second Edition. Edited by Edward A. Greenfield, Dana-Farber Cancer Institute, @2014. The present disclosure is not necessarily limited to any particular source, method of production, or other special characteristics of an antibody.

A therapeutic molecule may be an antisense oligonucleotide (ASO). In general, antisense oligonucleotides block the translation of a target protein by hybridizing to an mRNA sequence encoding the target protein, thereby inhibiting protein synthesis by ribosomal machinery. In some embodiments, the antisense oligonucleotide (ASO) targets a gene comprising a microsatellite repeat sequence. In some embodiments, the antisense oligonucleotide inhibits translation of one or more RAN proteins. One skilled in the art would understand how to construct an anti-sense oligonucleotide comprising a short (approximately 15 to 30 nucleotides) with a base sequence complementary to the RAN mRNA. One skilled in the art will understand that complementarity to the RAN mRNA can be established using canonical nucleotides comprising ribose, phosphate and one of the bases adenine, guanine, cytosine, and uracil linked with the phosphodiester linkages typifying naturally occurring nucleic acids OR some of the nucleotides could be modified by replacing the ribose with an alternate saccharide moiety such as 2′-deoxyribose, or 2′-O-(2-mehtoxyethyl)ribose, AND/OR some or all of the nucleotides could be modified by methylation, AND/OR some or all of the phosphodiester bonds between the nucleotides could be replaced with phosphorothioate linkages. Those skilled in the art will understand that modifications of several nucleotides at both the 3′ and 5′ ends of the antisense oligonucleotide to inhibit degradation by ubiquitous terminally active RNA nucleases will improve the stability and thus half-life of the antisense oligo. However, those skilled in the art will appreciate that it is also desirable that at least some part of the antisense oligo will, once complexed with the RAN mRNA promote the activity of Ribonuclease H to promote the enzymatic degradation of the RAN mRNA once it is complexed with the antisense oligo.

In some embodiments, the therapeutic agent is an inhibitory nucleic acid. In some embodiments, the inhibitory nucleic acid is an interfering RNA selected from the group consisting of dsRNA, siRNA, shRNA, miRNA, and ami-RNA. In some embodiments, the inhibitory nucleic acid is a nucleic acid aptamer (e.g., an RNA aptamer or DNA aptamer). Generally, an inhibitory RNA molecule can be unmodified or modified. In some embodiments, an inhibitory RNA molecule comprises one or more modified oligonucleotides, e.g., phosphorothioate-, 2′-O-methyl-, etc.-modified oligonucleotides, as such modifications have been recognized in the art as improving the stability of oligonucleotides in vivo.

In some embodiments, a therapeutic agent is an effective amount of a eukaryotic initiation factor 2 (eIF2) inhibiting agent or a Protein Kinase R (PKR) inhibiting agent (e.g., an inhibitor of eIF2 and/or PKR). In some embodiments, an inhibitor of eIF2 is an inhibitor of a serine/threonine kinase. Examples of serine/threonine kinases include but are not limited to protein kinase A (PKA), protein kinase C (PKC), Mos/Raf kinases, mitogen-activated protein kinases (MAPKs), protein kinase B (AKT kinase), etc. In some embodiments, an eIF2 inhibitor is a protein kinase R (PKR) inhibitor. Inhibitors of eIF2 and PKR are described, for example in International Application Publication No. WO 2018/195110, the entire content of which is incorporated herein by reference.

In some embodiments, the therapeutic agent is a protein kinase R (PKR) variant that functions in a dominant negative manner to inhibit phosphorylation of eIF2α. As used herein, “protein kinase R (PKR) variant” refers to a protein comprising an amino acid sequence that is at least 70%, at least 80%, at least 90%, at least 95%, at least 96%, at least 97%, at least 98%, or at least 99% identical to a wild-type protein kinase R (PKR) (e.g., GenBank Accession No. NP_002750.1), wherein the variant protein comprises at least one amino acid variation (also referred to sometimes as “mutation”) relative to the amino acid sequence of the wild-type PKR.

In some embodiments, the amino acid sequence of a PKR variant is at least 75%, at least 85%, at least 95%, at least 96%, at least 97%, at least 98%, at least 99%, at least 99.5%, at least 99.6%, at least 99.7%, at least 99.8%, or at least 99.9% identical to the amino acid sequence of wild-type PKR. In some embodiments, the amino acid sequence is about 95-99.9% identical to the amino acid sequence of wild-type PKR. In some embodiments, the protein comprises at least 1, at least 2, at least 3, at least 4, at least 5, at least 6, at least 7, at least 8, at least 9, at least 10, at least 11, at least 12, at least 13, at least 14, or at least 15 different amino acid sequence variations as compared to the sequence of amino acids set forth in the amino acid sequence of wild-type PKR. In some embodiments, a PKR variant comprises a mutation at position 296 (e.g., position 296 of a human wild-type PKR). In some embodiments, the mutation at position 296 is K296R.

An eIF2 inhibitor may be a direct inhibitor or an indirect inhibitor. Generally, a direct modulator functions by interacting with (e.g., interacting with or binding to) a gene encoding eIF2 (or eIF2α), or an eIF2 protein complex. Generally, an indirect modulator functions by interacting with a gene or protein that regulates the expression or activity of eIF2 or an eIF2α (e.g., does not directly interact with a gene or protein encoding eIF2 or an eiF2α).

In some embodiments, an inhibitor eIF2 or PKR is a selective inhibitor. A “selective inhibitor” refers to an inhibitor of eIF2 or PKR that preferentially inhibits activity or expression of one type of eIF2 subunit compared with other types of eIF2 subunits, or inhibits activity or expression of PKR preferentially compared to other kinases. In some embodiments, an inhibitor of eIF2 is a selective inhibitor of eIF2α. In some embodiments, an inhibitor of eIF2 is a selective inhibitor of eIF2A. In some embodiments, an inhibitor of eIF2 is a selective inhibitor of protein kinase R (PKR), such as a selective PKR inhibitor.

Examples of proteins that inhibit eiF2 (e.g., an eIF2 subunit) include but are not limited to polyclonal anti-eIF2 antibodies, monoclonal anti-eIF2 antibodies, etc. Examples of nucleic acid molecules that inhibit eiF2 (e.g., an eIF2 subunit) include but are not limited to dsRNA, siRNA, miRNA, etc. that target a gene encoding an eIF2 subunit (e.g., a gene encoding the mRNA set forth in GenBank Accession No. NM_004094.4). Examples of small molecule inhibitors of eIF2 include but are not limited to LY 364947, eIF-2α Inhibitor II Sal003, etc.

Examples of proteins that inhibit PKR include but are not limited to certain dominant negative PKR variants (e.g., K296R PKR mutant), TARBP2, etc. Examples of nucleic acid molecules that inhibit PKR include but are not limited to dsRNA, siRNA, miRNA, etc. that target a gene encoding a PKR. Examples of small molecule inhibitors of PKR include but are not limited to 6-amino-3-methyl-2-oxo-N-phenyl-2,3-dihydro-1H-benzo[d]imidazole-1-carboxamide, N-[2-(1H-indol-3-yl)ethyl]-4-(2-methyl-1H-indol-3-yl)pyrimidin-2-amine, metformin, buformin, phenformin, etc.

Examples of nucleic acid molecules that inhibit eIF2A include but are not limited to dsRNA, siRNA, miRNA, etc. that target a gene encoding an eIF2A (e.g., a gene encoding the mRNA set forth in GenBank Accession No. NM_032025.4). Examples of small molecule inhibitors of eIF2A include but are not limited to salubrinal, Sal003, ISRIB, etc.

In some embodiments, the eIF2 inhibitor or PKR inhibitor is an interfering (e.g., inhibitory) nucleic acid. In some embodiments, the inhibitory nucleic acid is an interfering RNA selected from the group consisting of dsRNA, siRNA, shRNA, mi-RNA, and ami-RNA. In some embodiments, the inhibitory nucleic acid is an antisense nucleic acid (e.g., an antisense oligonucleotide (ASO) or a nucleic acid aptamer (e.g., an RNA aptamer). Generally, an inhibitory RNA molecule can be unmodified or modified. In some embodiments, an inhibitory RNA molecule comprises one or more modified oligonucleotides, e.g., phosphorothioate-, 2′-O-methyl-, etc.-modified oligonucleotides, as such modifications have been recognized in the art as improving the stability of oligonucleotides in vivo.

In some embodiments, the interfering RNA comprises a sequence that is complementary with between 5 and 50 continuous nucleotides (e.g., 5, 6, 7, 8, 9, 10, 11, 12, 13, 14, 15, 16, 17, 18, 19, 20, 21, 22, 23, 24, 25, about 30, about 35, about 40, or about 50 continuous nucleotides) of a nucleic acid sequence (such as an RNA sequence) encoding an eIF2 subunit or a nucleic acid sequence (such as an RNA sequence) encoding PKR.

In some embodiments, a therapeutic agent is an inhibitor of Eukaryotic initiation factor 3 (eIF3), which is a multiprotein complex that is involved with the initiation phase of eukaryotic protein translation. Generally, in humans eIF3 comprises 13 non-identical subunits (e.g., eIF3a-m). Mammalian eIF3, the largest most complex initiation factor, comprises up to 13 non-identical subunits. Typically, eIF3f is involved in many steps of translation initiation including stabilization of the ternary complex, mediating binding of mRNA to 40S subunit and facilitating dissociation of 40S and 60S ribosomal subunits. In some embodiments, therapeutic agents that inhibit expression or activity of an eIF3 subunit (e.g., eIF3f, eIF3m, eIF3h, or other eIF3 subunit) can be used to reduce or inhibit RAN translation in a cell or in a subject (e.g., a subject having Alzheimer's disease characterized by RAN protein translation). Inhibitors of eIF3 subunits are further described, for example in International Application Publication No. WO 2017/176813, the entire content of which is incorporated herein by reference.

An eIF3 inhibitor may be a direct inhibitor or an indirect inhibitor. Generally, a direct modulator functions by interacting with (e.g., interacting with or binding to) a gene encoding eIF3 (or an eIF3 subunit), or an eIF3 protein complex, or an eIF3 subunit. Generally, an indirect modulator functions by interacting with a gene or protein that regulates the expression or activity of eIF3 or an eIF3 subunit (e.g., does not directly interact with a gene or protein encoding eIF3 or an eiF3 subunit). In some embodiments, an inhibitor of eIF3 is a selective inhibitor. A “selective inhibitor” refers to a modulator of eIF3 that preferentially inhibits activity or expression of one type of eIF3 subunit compared with other types of eIF3 subunits. In some embodiments, an inhibitor of eIF3 is a selective inhibitor of eIF3f.

An eIF3 inhibitor can be a protein (e.g., antibody), nucleic acid, or small molecule. Examples of proteins that inhibit eiF3 (e.g., an eIF3 subunit) include but are not limited to polyclonal anti-eIF3 antibodies, monoclonal anti-eIF3 antibodies, Measles Virus N protein, Viral stress-inducible protein p56, etc. Examples of nucleic acid molecules that inhibit eiF3 (e.g., an eIF3 subunit) include but are not limited to dsRNA, siRNA, miRNA, amiRNA, etc. that target a gene encoding an eIF3 subunit. Examples of small molecule inhibitors of eIF3 include but are not limited to mTOR inhibitors (e.g., rapamycin, PP242), S6 kinase (S6K) inhibitors, etc.

In some embodiments, an interfering RNA comprises a sequence that is complementary with between 5 and 50 continuous nucleotides (e.g., 5, 6, 7, 8, 9, 10, 11, 12, 13, 14, 15, 16, 17, 18, 19, 20, 21, 22, 23, 24, 25, about 30, about 35, about 40, or about 50 continuous nucleotides) of a nucleic acid sequence (such as an RNA sequence) encoding an eIF3 subunit. Examples of nucleic acid sequences encoding eIF3 subunits include GenBank Accession No. NM 003750.2 (eIF3a), GenBank Accession No. NM_003751.3 (eIF3b), GenBank Accession No. NM_003752.4 (eIF3c), GenBank Accession No. NM_003753.3 (eIF3d), GenBank Accession No. NM_001568.2 (eIF3e), GenBank Accession No. NM_003754.2 (eIF3f), GenBank Accession No. NM_003755.4 (eIF3g), GenBank Accession No. NM_003756.2 (eIF3h), GenBank Accession No. NM_003757.3 (eIF3i), GenBank Accession No. NM_003758.3 (eIF3j), GenBank Accession No. NM_013234.3 (eIF3k), GenBank Accession No. NM_016091.3 (eIF3l), GenBank Accession No. NM_006360.5 (eiF3m), etc. In some embodiments, the interfering RNA is a siRNA. In some embodiments, an eIF3f siRNA is administered (e.g., Dharmacon Cat #J-019535-08). In some embodiments, an eIF3m siRNA is administered (e.g., Dharmacon Cat #J-016219-12). In some embodiments, an eIF3h siRNA is administered (e.g., Dharmacon Cat #J-003883-07).

In some embodiments, eIF3f is a negative regulator of RAN translation and decreased levels of human eIF3f are associated with decreased accumulation of RAN protein in cells. In some embodiments, RAN translation (e.g., in cells expressing a RAN protein) is sensitive to eIF3f knockdown unlike translation from close cognate or AUG translation. In some embodiments, the translational machinery used for RAN translation is distinct from AUG and near AUG translation machinery in a cell.

In some embodiments, a therapeutic agent is an inhibitor of TLR3. An inhibitor of TLR3 can be a protein (e.g., antibody), nucleic acid, or small molecule. Examples of proteins that inhibit TLR3 include but are not limited to polyclonal anti-TLR3 antibodies, monoclonal anti-TLR3 antibodies, etc. Examples of nucleic acid molecules that inhibit TLR3 include but are not limited to dsRNA, siRNA, miRNA, amiRNA, etc. that target a gene encoding TLR3. Examples of small molecule inhibitors of TLR3 are described, for example in Cheng et al. (2011) J Am Chem Soc 133 (11): 3764-7.

In some embodiments, a therapeutic agent is an inhibitor of p62 protease. An inhibitor of p62 can be a protein (e.g., antibody), nucleic acid, or small molecule. Examples of proteins that inhibit p62 include but are not limited to polyclonal anti-p62 antibodies, monoclonal anti-p62 antibodies, etc. Examples of nucleic acid molecules that inhibit p62 include but are not limited to dsRNA, siRNA, miRNA, amiRNA, etc. that target a gene encoding p62. In some embodiments, a therapeutic agent is an agent that increases proteasome activity, for example as described in Leestemaker et al. (2017) Cell Chemical Biology 24, 725-736.

In some embodiments, a therapeutic agent comprises a peptide antigen that targets one or more RAN proteins (e.g., is a RAN protein vaccine that targets one or more RAN proteins). In some embodiments, the peptide antigen targets (e.g., comprises an amino acid sequence encoding) one or more of the RAN proteins poly(PR) poly(GR); poly(GP); and poly(GA).

In some embodiments, one or more therapeutic molecules are administered to a subject to treat a disease associated with RAN proteins characterized by an expansion of a nucleic acid repeat (e.g., associated with a repeat associated non-ATG translation). For example, in some embodiments, a subject is administered 2, 3, 4, 5, 6, 7, 8, 9, or 10 therapeutic agents (e.g., proteins, nucleic acids, small molecules, etc., or any combination thereof).

### Administration

A therapeutic agent may be delivered by any suitable modality known in the art. In some embodiments, a therapeutic agent (e.g., a protein, antibody, interfering nucleic acid, etc.) is delivered to a subject by a vector, such as a viral vector (e.g., adenovirus vector, recombinant adeno-associated virus vector (rAAV vector), lentiviral vector, etc.) or a plasmid-based vector. In some embodiments, a therapeutic agent is delivered to a subject (e.g., a subject having C9− sALS characterized by expression of one or more RAN proteins) in a recombinant adeno-associated virus (rAAV) particle.

In some embodiments, a recombinant rAAV particle comprises a nucleic acid vector, such as a single-stranded (ss) or self-complementary (sc) AAV nucleic acid vector. In some embodiments, the nucleic acid vector comprises a transgene encoding a therapeutic agent as described herein (e.g., a protein, antibody, interfering nucleic acid, etc.), and one or more regions comprising inverted terminal repeat (ITR) sequences (e.g., wild-type ITR sequences or engineered ITR sequences) flanking the expression construct. In some embodiments, the nucleic acid is encapsidated by a viral capsid. In some embodiments, the transgene is operably linked to a promoter, for example a constitutive promoter or an inducible promoter. In some embodiments, the promoter is a tissue-specific (e.g., CNS-specific) promoter. In some embodiments, a rAAV particle comprises a viral capsid that has a tropism for CNS tissue, for example AAV9 capsid protein or AAV.PHPB capsid protein.

Aspects of the disclosure relate to the delivery of a therapeutically effective amount of a therapeutic agent to a subject. In some embodiments, a therapeutically effective amount is an amount effective in reducing repeat expansions in the subject. In some embodiments, a therapeutically effective amount is an amount effective in reducing the transcription of RNAs that produce RAN proteins in a subject. In certain embodiments, a therapeutically effective amount is an amount effective in reducing the translation of RAN proteins in a subject. In some embodiments, a therapeutically effective amount is an amount effective for treating C9− sALS. “Reducing” expression of a repeat sequence or RAN protein translation refers to a decrease in the amount or level of repeat sequence expression or RAN protein translation in a subject after administration of a therapeutic agent (and relative to the amount or level in the subject prior to the administration).

In certain embodiments, the effective amount is an amount effective in reducing the level of RAN proteins by at least 10%, at least 20%, at least 30%, at least 40%, at least 50%, at least 60%, at least 70%, at least 80%, at least 90%, at least 95%, or at least 98% (e.g., the level of RAN proteins relative to the level of RAN proteins in a cell or subject that has not been administered a therapeutic agent). In certain embodiments, the effective amount is an amount effective in reducing the translation of RAN proteins by at least 10%, at least 20%, at least 30%, at least 40%, at least 50%, at least 60%, at least 70%, at least 80%, at least 90%, at least 95%, or at least 98% (e.g., the level of RAN proteins relative the level of RAN proteins in a cell or subject that has not been administered a therapeutic agent).

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof. The exemplary liquid dosage forms in certain embodiments are formulated for ease of swallowing, or for administration via feeding tube.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinyl pyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Therapeutic agents described herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

A therapeutic agent can be administered by any route, including enteral (e.g., oral), parenteral, intravenous, intramuscular, intra-arterial, intramedullary, intrathecal, subcutaneous, intraventricular, transdermal, interdermal, ocular, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Systemic routes include oral and parenteral. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a therapeutic agent required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of a compound described herein. In certain embodiments, when multiple doses are administered to a subject or applied to a biological sample, tissue, or cell, the frequency of administering the multiple doses to the subject or applying the multiple doses to the biological sample, tissue, or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample, tissue, or cell is one dose per day. In certain embodiments, the frequency of administering the multiple doses to the subject or applying the multiple doses to the biological sample, tissue, or cell is two doses per day. In certain embodiments, the frequency of administering the multiple doses to the subject or applying the multiple doses to the biological sample, tissue, or cell is three doses per day. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, eight months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

In some embodiments, a treatment for a disease associated with RAN protein expression is administered to the central nervous system (CNS) of a subject in need thereof. As used herein, the “central nervous system (CNS)” refers to all cells and tissues of the brain and spinal cord of a subject, including but not limited to neuronal cells, glial cells, astrocytes, cerebrospinal fluid, etc. Modalities of administering a therapeutic agent to the CNS of a subject include direct injection into the brain (e.g., intracerebral injection, intraventricular injection, intraparenchymal injection, etc.), direct injection into the spinal cord of a subject (e.g., intrathecal injection, lumbar injection, etc.), or any combination thereof.

In some embodiments, a treatment as described by the disclosure is systemically administered to a subject, for example by intravenous injection. Systemically administered therapeutic molecules can be modified, in some embodiments, in order to improve delivery of the molecules to the CNS of a subject. Examples of modifications that improve CNS delivery of therapeutic molecules include but are not limited to co-administration or conjugation to blood brain barrier-targeting agents (e.g., transferrin, melanotransferrin, low-density lipoprotein (LDL), angiopeps, RVG peptide, etc., as disclosed by Georgieva et al. Pharmaceuticals 6(4): 557-583 (2014)), coadministration with BBB disrupting agents (e.g., bradykinins), and physical disruption of the BBB prior to administration (e.g., by MRI-Guided Focused Ultrasound), etc.

The following Examples are intended to illustrate the benefits of the present invention and to describe particular embodiments, but are not intended to exemplify the full scope of the invention. Accordingly, it will be understood that the Examples are not meant to limit the scope of the invention.

### EXAMPLES

### Example 1

This example describes the pathological identification of RAN proteins in biological samples obtained from subjects having genetically unknown (e.g., C9orf72 and/or SCA36 negative) sporadic amyotrophic lateral sclerosis (C9− sALS), and the identification of certain genes which may contribute to the expression of said RAN proteins. These RAN proteins may contribute to disease either from single unidentified repeat expansion mutations or from the combined effects of multiple genes, each with smaller pre-mutation lengths. Data indicates that repeat RNAs and/or RAN proteins produced from these expansion mutations contribute to disease by disrupting protein homeostasis, proteasome function and autophagy.

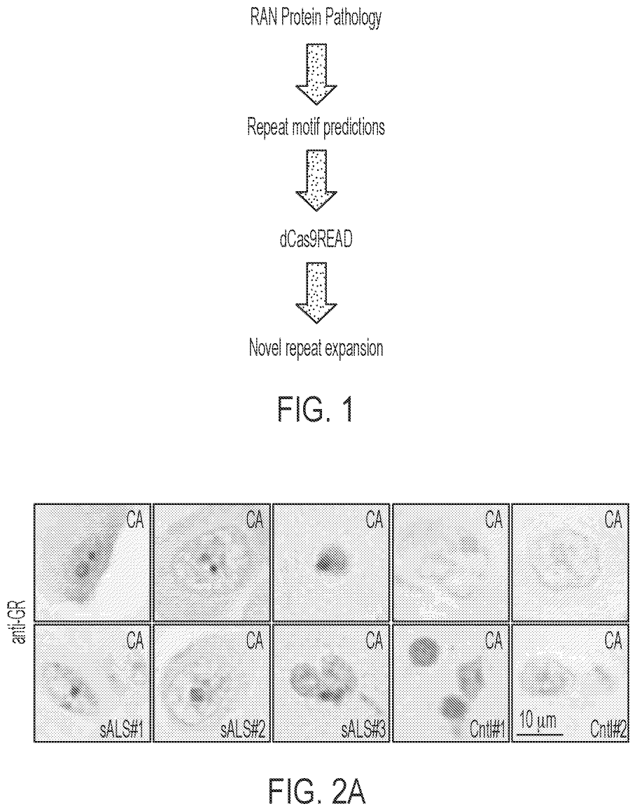

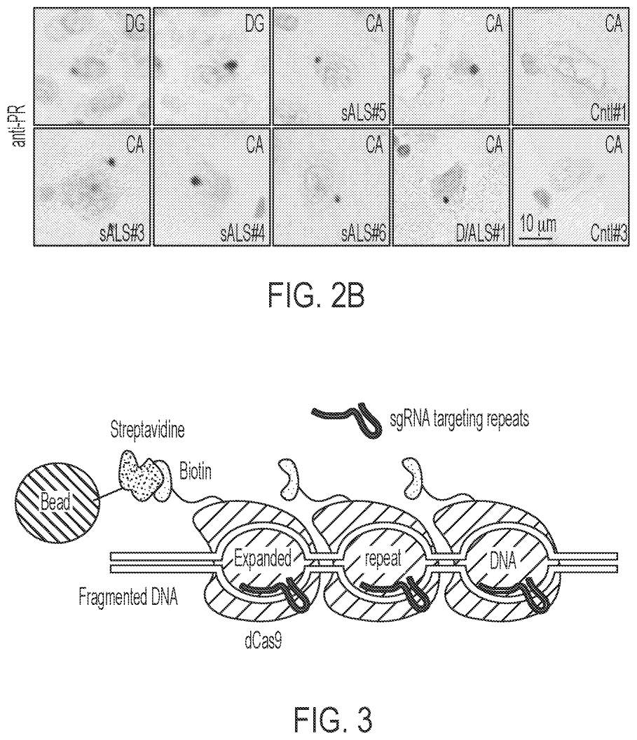

Briefly, immunohistochemical staining (IHC) was performed on brain samples obtained from subjects having sALS of unknown genetic origin (see FIG. 1). Subjects were confirmed to not express a repeat mutation within the C9orf72 or SCA36 genes. Samples were stained for GR (FIG. 2A), PR (FIG. 2B), GA, and/or GP aggregates, and were characterized as being either positive (+) or negative (−) for each repeat. Results for all C9− sALS samples are shown in Table 5. Of the 34 samples tested for GA and GP aggregates, 32% (11/34) were positive for GA aggregates and 12% (4/34) were positive for GP aggregates. Of the 33 samples tested for PR aggregates, 79% (26/33) were positive for PR aggregates. Of the 10 samples tested for GR aggregates, 30% (3/10) were positive for GR aggregates.

TABLE 5
Detection of RAN proteins in sALS autopsy samples

| | RAN proteins | | | |
|---|---|---|---|---|
| Sample | poly(GA) | | | |
| No. | (−/+) | poly(GR) (−/+) | poly(PR) (−/+) | poly(GP) (−/+) |
| 1 | − | + | + | − |
| 2 | + | + | + | − |
| 3 | + | − | + | − |
| 4 | + | nt | + | − |
| 5 | + | nt | + | − |
| 6 | − | nt | − | − |
| 7 | + | nt | + | − |
| 8 | − | nt | + | − |
| 9 | − | nt | − | − |
| 10 | + | nt | + | + |
| 11 | + | nt | + | − |
| 12 | − | nt | + | − |
| 13 | − | nt | − | − |
| 14 | − | nt | + | − |
| 15 | − | − | + | − |
| 16 | − | nt | + | − |
| 17 | − | nt | + | − |
| 18 | + | − | + | − |
| 19 | − | nt | + | − |
| 20 | − | nt | + | + |
| 21 | − | nt | + | − |
| 22 | − | − | + | − |
| 23 | − | + | + | + |
| 24 | − | nt | + | − |
| 25 | − | − | nt | − |
| 26 | − | − | − | − |
| 27 | − | − | + | − |
| 28 | − | nt | + | − |
| 29 | − | nt | − | − |
| 30 | + | nt | + | − |
| 31 | + | nt | − | + |
| 32 | − | nt | + | − |
| 33 | − | nt | + | − |
| 34 | + | nt | − | − |

nt = not tested;
+ = sALS sample was positive for indicated RAN protein;
− = sALS sample was negative for indicated RAN protein

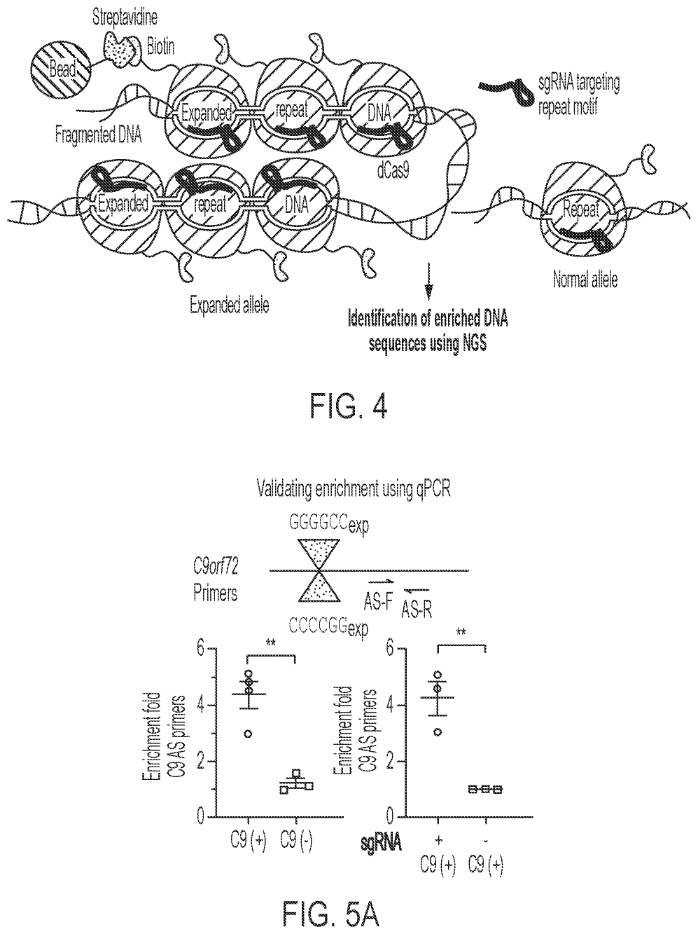

Following the IHC analysis, the repeat motifs of the putative C9− sALS RAN proteins were used to identify all possible DNA sequences that could encode the RAN proteins. For example, possible DNA sequences encoding PR, GR, GA, and GP are shown in Tables 1-4. The identified DNA sequences were used to develop sgRNAs which were combined with biotin-tagged nuclease-deficient Cas9 (dCas9) to pull down and enrich for the repeat expansion mutations and corresponding flanking sequences from genomic DNA isolated from C9− sALS tissue samples positive for RAN proteins. Expanded repeats provide multiple binding sites for sgRNAs, thus increasing the probability of interaction between sgRNA-dCas9 complexes and expanded repeats compared to shorter repeat tracts (FIG. 3).

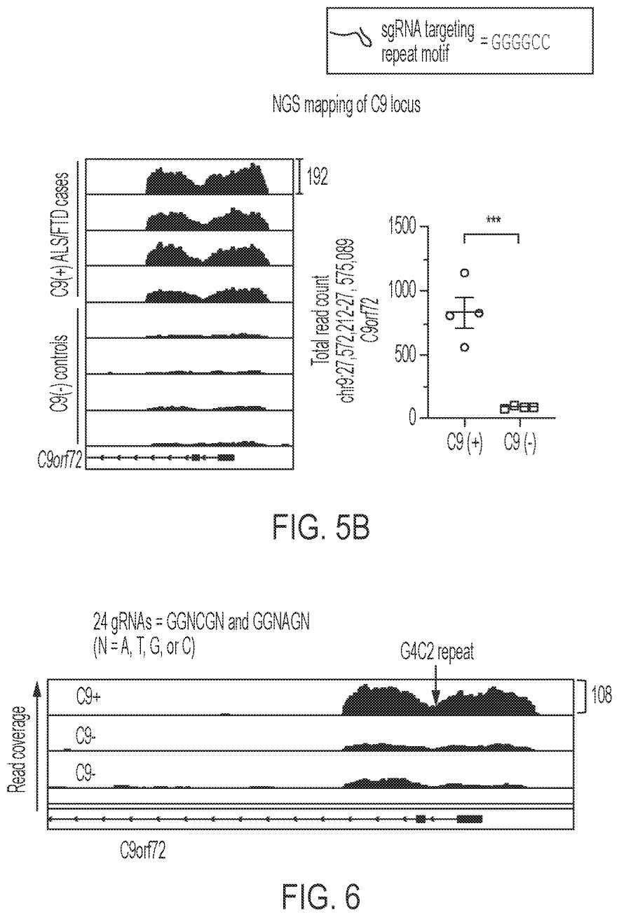

This dCas9-based detection and enrichment tool (dCas9READ, e.g., as described in International Publication No. WO 2021/007110 A1, incorporated by reference herein in its entirety) pulls down and enriches specific DNA sequences by taking advantage of the rapid kinetics and high stability of single guide RNA/dCas9 (sgRNA-dCas9) complexes without the need to denature target DNA (FIG. 4). Favored binding of sgRNA-dCas9 complexes to repeat expansion allows the enrichment and the identification of the repeat and unique flanking sequence. Repeat expansions were pulled-down, and repeat expansions and their unique flanking sequences were identified using next generation sequencing (NGS).

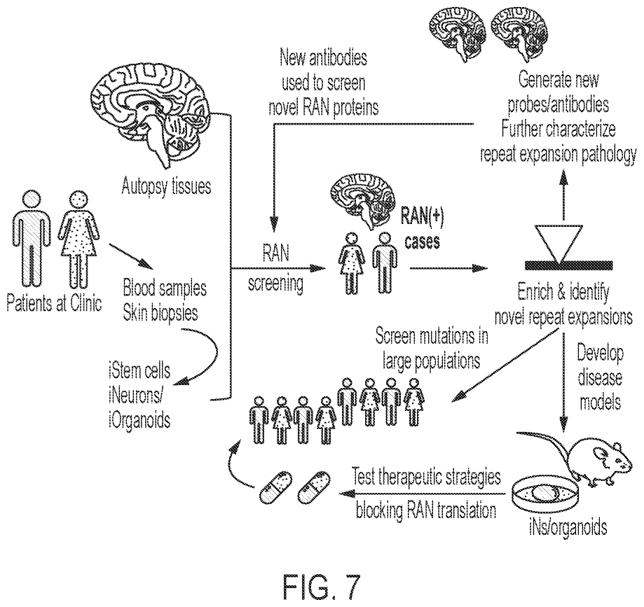

As proof-of concept, the dCas9READ method described herein was used to isolate a GGGGCC (G4C2) repeat expansion motif. While the G4C2 repeat expansion motif is associated with cases of C9+ ALS, it can also be associated with C9-ALS. The dCas9READ enrichment method was validated using quantitative polymerase chain reaction (qPCR) (FIG. 5A). Sequencing of the upstream and downstream regions flanking the repeat was used to identify the specific location of the repeat expansion (FIG. 5B).

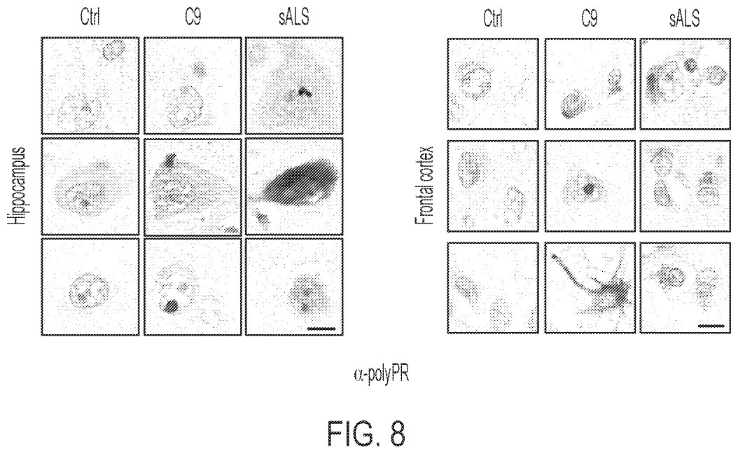

sgRNAs can be developed using the nucleic acid sequences encoding each type of di-peptide repeat motif (e.g., as shown in Tables 1-4). Notably, the dCas9READ method described herein can be used with a mixture of sgRNAs, and multiple repeats can be screened simultaneously using dCas9READ. FIG. 6 shows representative data demonstrating these capabilities of the dCas9READ method described herein.

Table 6 shows genes comprising RAN protein-encoding repeat expansion motifs which were identified using the dCas9READ method described herein. These genes may contribute to the observed expression of RAN proteins in C9− sALS autopsy samples.

TABLE 6
List of enriched genes
Gene Name

ABCB4
ABCB5
ABCC6
ABHD17C
ABHD6
ACHE
ACTA1
ACTL8
ADAMTS14
ADIPOQ-
AS1
AGAP12P
AGAP9
AHSA2P
AIFM2
AIG1
ALOX5
ANHX
ANK3
ANKRD20A9P
ANKRD33
ANKRD33B
ANKRD35
ANKRD50
ANKRD62
ANKRD7
ANXA8L1
AP1S2
APOOL
ARHGAP12
ARMCX4
ARPC1B
ASGR1
ASIC1
ASXL3
AUTS2
AVPR2
B3GNT7
BAHD1
BANP
BARD1
BARHL2
BCL11B
BEND4
BMS1P1
BRMS1L
BRSK2
BSN
BUD13
C16orf78
C16orf95
C1orf61
C5orf22
C9orf139
C9orf50
C9orf72
CACNA2D2
CALCOCO1
CALU
CASZ1
CBX7
CCM2L
CCNI
CCNY
CD79B
CD82
CDC42
CDC42BPB
CDC42EP1
CDH4
CDK5R1
CDKN1C
CDON
CDYL
CEBPB
CHD7
CHEK2P2
CHGA
CHID1
CHMP6
CIT
CITED4
CLASP 2
CLCF1
CLUH
COL24A1
COL26A1
COLEC11
COX7A1
CPLX1
CPNE1
CPNE7
CRAMP1
CRELD2
CRIPAK
CRNDE
CROCC
CUX1
CXADRP 3
CXorf36
CXXC5
CYP4F29P
CYP4F35P
DAB2IP
DAP
DCP1B
DDX11L1
DDX11L9
DGCR8
DLGAP 4
DMD
DNAH14
DNAH9
DNAJC5G
DNM1P35
DNM1P46
DOCK3
DOCK5
DOT1L
DPP6
DUSP 2
DUX4L8
EDARADD
EIF6
ELK2AP
EN2
ERICH6B
EVL
EXOC3-AS1
EXOC6B
FAM110C
FAM149A
FAM230C
FAM27B
FAM27C
FAM53B
FAM95A
FER
FGF17
FGFR4
FGFRL1
FKBP8
FLNA
FNDC3A
FOXK2
FOXN3
FRAS1
FRG1BP
FTMT
FXYD7
GABRB2
GALNT9
GAP43
GCAT
GID8
GKAP1
GLP1R
GNG4
GPHN
GPR137B
GPR52
GPSM1
GS1-
124K5.11
GSTM3
HAAO
HAUS7
HSPB1
HTT
HUWE1
HYDIN2
ICE1
IFITM10
IGHMBP 2
IL13RA1
IL9R
IPO5
IQSEC1
IRX1
IRX4
ITGB1
ITPR2
KBTBD2
KCNQ2
KIAA1024
KIF25
KLF13
KMT5C
KNDC1
KRT8
LARGE1
LARP4B
LATS2
LHFPL4
LINC00051
LINC00266-
1
LINC00311
LINC00443
LINC00482
LINC00486
LINC00494
LINC00668
LINC00840
LINC00937
LINC00938
LINC01193
LINC01300
LINC01310
LINC01588
LINC01881
LINC02291
LMO1
LMX1B
LOC100129534
LOC100134317
LOC100240735
LOC100505658
LOC100506023
LOC200772
LOC283683
LOC389831
LOC440434
LOC441081
LOC442497
LOC643201
LOC728743
LRMDA
LRP1
LRRC4B
LRRC4C
LTBP4
LYST
MAD1L1
MAPK11
MARCHF4
MBD2
MCF2L
MCF2L-AS1
MCL1
MEA1
MED16
MEGF8
METAP1
METRNL
METTL9
MGMT
MIR1208
MIR1257
MIR126
MIR1292
MIR1324
MIR1976
MIR3156-1
MIR3188
MIR4305
MIR4444-1
MIR4473
MIR4486
MIR4492
MIR4743
MIR4758
MIR4762
MIR5189
MIR548I1
MIR644A
MIR662
MLLT10
MMEL1
MOB2
MOCS2
MPRIP
MRPL12
MRPS34
MTDH
MTG1
MUC1
MX1
MYL10
MYO16-AS1
MYO5A
MYOZ3
NAA10
NADSYN1
NAGPA-
AS1
NAT10
NBAT1
NCF1B
NCKAP5
NEO1
NFATC1
NPC1L1
NPEPPS
NPRL3
NPTXR
NRG3
NT5E
NTNGI
NTSR1
NTSR2
NUDT16L1
NUMB
OGFR
OLIG2
OSGIN1
OSTN
P4HA2
PACRG
PAF1
PARP1
PARP14
PAX6
PAXIP1
PBX3
PCDH10
PCMT1
PDE4C
PDE4DIP
PDGFA
PDGFRA
PDS5A
PEX14
PGBD2
PHOSPHO1
PICALM
PICSAR
PIGZ
PIMREG
PITX1
PLEKHG4B
PLEKHM2
PLXNA1
PNOC
PODXL
POLR3B
POLRMT
POMC
POU6F1
PPARG
PPARGC1B
PPP1R1A
PRKAG2
PRMT2
PRNP
PRRX2
PRTFDC1
PTH1R
PTPN18
PTPRF
PTPRG
RAB20
RAD18
RAP1GAP
RASGEF1A
RBM5-AS1
RCBTB1
REPS1
RFLNA
RFPL4AL1
RHBDF1
RIMS1
RNA5-
8SN5
RNF34
ROCK1P1
RPL10
RPL23AP87
RPL29
RPLP1
RPS6KC1
RRP 36
RRS1-AS1
RYK
RYR1
SALL1
SBF1
SBK1
SCAF1
SCNN1D
SDCBP
SEC63
SELENOM
SERF1A
SERPINH1
SESN3
SH3BP4
SHARPIN
SHISA8
SHROOM2
SIK1
SIN3A
SIPA1L3
SIX5
SLC17A5
SLC19A1
SLC34A1
SLC44A3
SLC46A3
SLC6A6
SMAD3
SMIM24
SMOC2
SMPD1
SMUG1
SNORA80E
SNORD17
SNTG2
SOX1
SOX13
SOX17
SPATA32
SRC
ST6GALNAC6
STAG3L4
STARD8
STK39
STON2
STX10
STX17-AS1
SULF2
SUPT20HL2
SYT2
TBC1D12
TBC1D3
TBC1D3B
TBC1D3C
TBC1D3H
TBCD
TCERG1
TDRD3
TFAP 2B
THAP4
THBS2
TIAF1
TIGAR
TLE4
TMEM121
TMEM150C
TNFSF12
TNK2
TNRC6B
TNS2
TOR1A
TPD52L1
TPRA1
TRAPPC8
TRIM29
TRPC2
TRPM6
TRPV5
TSPAN12
TSPAN5
TTLL5
TUBGCP4
UCN2
UGT8
ULK1
UNCX
UNK
UPK3A
USF2
VCL
VPS37C
WASF2
WDR4
WSCD1
XPR1
YWHAQ
ZBTB24
ZBTB7A
ZDHHC1
ZFYVE28
ZNF213
ZNF354C
ZNF37A
ZNF480
ZNF516
ZNF554
ZNF621
ZNF622
ZNF675

The data described in this example highlight methods to identify those repeat expansions that lead to the accumulation of RAN protein aggregates in C9− sALS brains. Antibodies against RAN protein repeat motifs are also used to screen human C9− sALS autopsy brains for RAN aggregates.

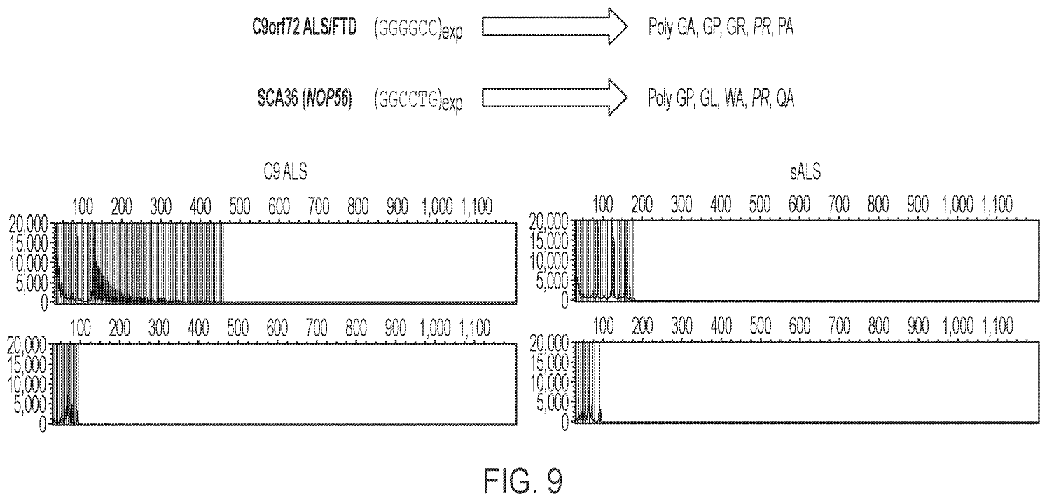

FIG. 7 shows an example of a pathology-to-genetics strategy for studying novel repeat expansion mutations, as described herein.

### Example 2

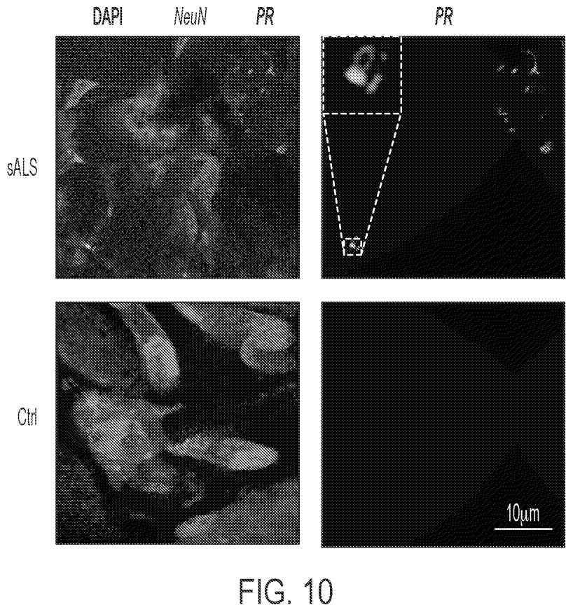

This example describes investigation of the presence of RAN proteins in sporadic ALS (sALS) cases that are negative for C9orf72 and SCA36 repeat expansions. FIG. 8 shows representative immunohistochemistry (IHC) data indicating that poly(PR) RAN proteins were present in 22% of sALS brain tissue samples tested.

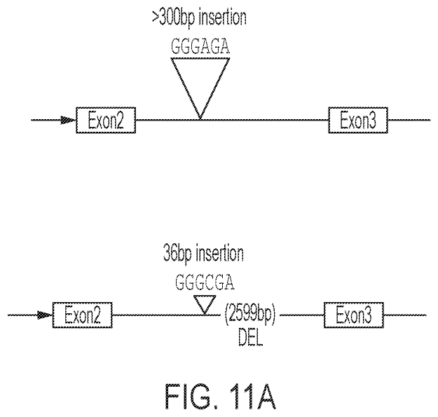

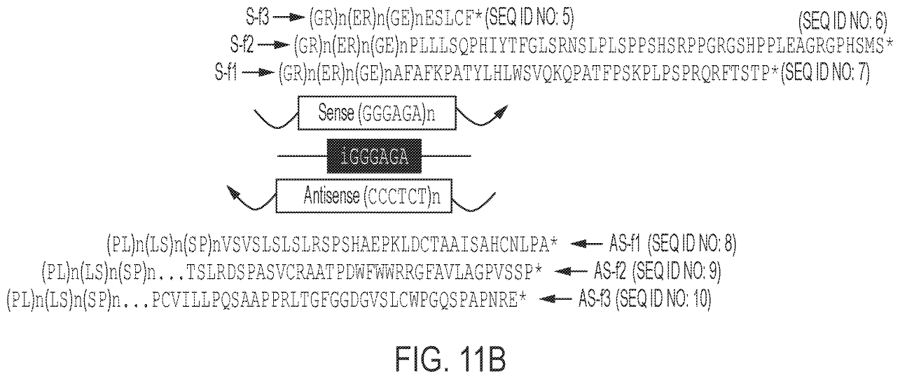

Aggregation patterns of poly(PR) differed between sALS and C9 ALS samples in both the hippocampus and frontal cortex samples. Gene expression analysis was performed and it was observed that poly(PR) sALS samples were negative for C9orf72 and SCA36 expansion repeats, indicating that poly(PR) RAN proteins found in sALS samples are expressed from novel repeat expansions (FIG. 9). Poly (PR) aggregates were also detected in sALS brain organoids generated from patient-derived iPSCs generated from blood samples (FIG. 10).

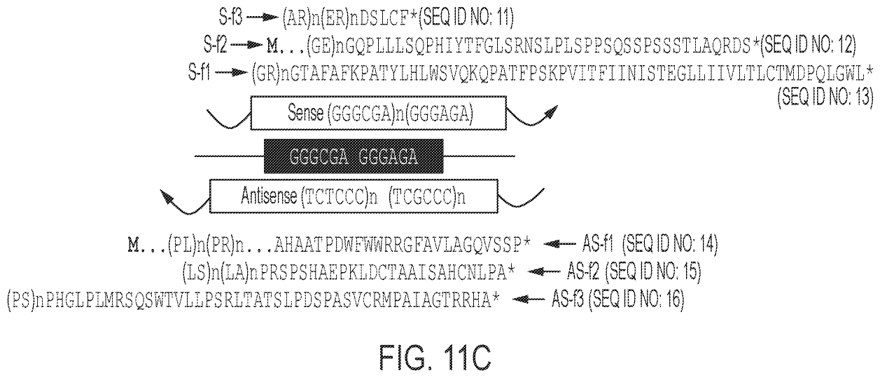

CRISPR deactivated Cas9 repeat enrichment and detection (dCas9READ) was used to identify novel candidate PR-encoding repeat expansions in genomic DNA from sALS patient samples. Data indicate that ADAM Metallopeptidase with Thromnospondin Type 1 Motif 14 (ADAMTS14) contains a repeat expansion encoding poly(GR) and poly(PR) RAN proteins. The encoded preproprotein of ADAMTS14 is proteolytically processed to generate the mature enzyme. This enzyme cleaves amino-terminal propeptides from type I procollagen, a necessary step in the formation of collagen fibers. Amino-procollagenase activity has been described for ADAMTS-2, -3, and -14, which suggests the requirement of these enzymes in the maturation and formation of collagen fibers within the extracellular matrix. Other members of ADAMTS family have been reported to be associated with stroke, inflammation and Alzheimer's Disease. Two repeat expansion configurations were identified between exons 2 and 3 of ADAMTS14 (FIG. 11). In one configuration, expanded alleles encode poly(GR) having between 40 and 70 repeats. In the other configuration, a depetion of approximately 2.6 kb or genomic DNA results in a poly(PR) RAN protein having ˜10 repeats.

FIG. 11B shows a schematic for poly(GR) RAN translation from an ADAMTS14 allele. Repeat expansions encoding poly(ER) and poly(GE) are also present in this repeat expansion in the sense direction, and poly(PL), poly(LS), and poly(SP) in the antisense direction. The unique C-terminal region for each expansion repeat is also shown.

FIG. 11C shows a schematic for poly(PR) RAN translation from an ADAMTS14 allele. Repeat expansions encoding poly(AR) and poly(ER), and poly(GE) are also present in this repeat expansion in the sense direction, and poly(PL), poly(LS), poly(PS), and poly(LA) in the antisense direction. The unique C-terminal region for each expansion repeat is also shown.

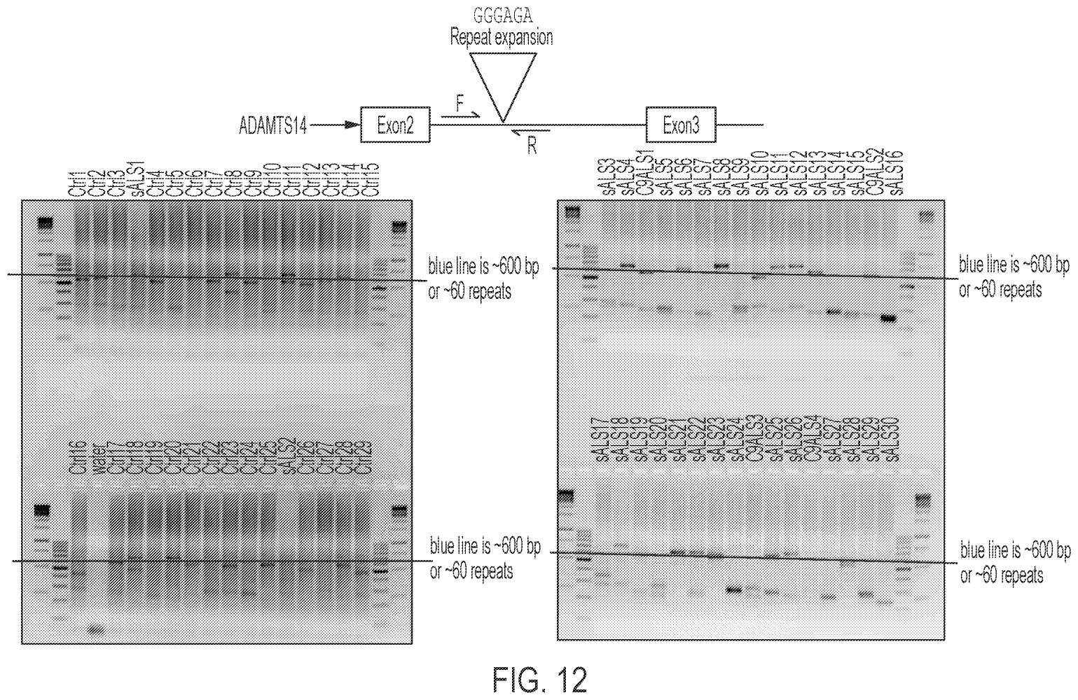

FIG. 12 shows representative gel images of sALS, C9orf72 ALS, and control samples with or without ADAMTS14 poly(GR)-encoding repeat expansions.

Patient samples having at least one allele with an expansion repeat of >600 bp were analyzed. In an unaffected (e.g., healthy) control group, 15/86 total samples (17.4%) were positive for an ADAMTS14 expansion repeat. In the sALS group, C9orf72 and SCA36 positive samples were excluded; 34/104 total samples (33%) were positive for ADAMTS14 expansion repeat. Odds ratios were calculated, and indicate that patients have a 2.2-fold increased risk of developing sALS when they have an ADAMTS14 poly(GR)-encoding expansion that is greater than or equal to 60 repeats in length.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A method comprising:
(i) obtaining a biological sample from a subject;
(ii) detecting in the biological sample obtained from the subject at least one RAN protein; and
(iii) when at least one RAN protein is detected in (ii), determining that the at least one RAN protein is not expressed from a C9orf72 or SCA36 locus of the subject.

2. A method comprising:
(i) detecting in a biological sample obtained from a subject at least one RAN protein;
(ii) when at least one RAN protein is detected in (i), determining that the at least one RAN protein is not expressed from a C9orf72 or SCA36 locus of the subject; and
(iii) identifying the subject has having or being at risk of developing sporadic ALS (sALS) based on the detecting of at least one RAN protein not expressed from a C9orf72 or SCA36 locus of the subject.

3. A method for diagnosing C9orf72 negative sporadic amyotrophic lateral sclerosis (C9− sALS), the method comprising:
(i) detecting in a biological sample obtained from a subject at least one RAN protein; and
(ii) diagnosing the subject as having C9− sALS based upon the presence of the at least one RAN protein.

4. The method of claim 3, wherein the at least one RAN protein is not expressed from a C9orf72 or SCA36 locus of the subject, optionally wherein the at least one RAN protein is expressed from an ADAMTS14 locus of the subject.

5. A method for diagnosing C9orf72 negative sporadic amyotrophic lateral sclerosis (C9− sALS), the method comprising:
(i) detecting in a biological sample obtained from a subject at least one RAN protein;
(ii) when at least one RAN protein is detected in (i), determining that the at least one RAN protein is not expressed from a C9orf72 locus of the subject; and
(iii) diagnosing the subject as having C9− sALS based on the presence of the at least one RAN protein that was not expressed from the C9orf72 locus.

6. The method of any one of claims 1-5, wherein the step of detecting comprises performing an assay on the biological sample.

7. The method of any one of claims 2-6, further comprising administering to the identified or diagnosed subject a therapeutic agent for the treatment of the sALS.

8. A method for treating C9orf72 negative sporadic amyotrophic lateral sclerosis (C9− sALS) in a subject, the method comprising:
(i) administering to the subject a therapeutic agent for the treatment of C9− sALS,
wherein the subject has been diagnosed as having C9− sALS according to the method of any one of claims 3-7.

9. The method of any one of claims 1-8, wherein the biological sample is blood, serum, or cerebrospinal fluid (CSF).

10. The method of any one of claims 1-9, wherein the subject is a mammalian subject, optionally a human subject or a mouse subject.

11. The method of any one of claims 1-10, wherein 1, 2, 3, or 4 RAN proteins are detected.

12. The method of any one of claims 1-11, wherein the at least one RAN protein is a poly(GP), poly(GA), poly(GR), and/or poly(PR) RAN protein.

13. The method of any one of claims 1-12, wherein the at least one RAN protein is encoded by a gene comprising between 2 and 10,000 repeats of a nucleic acid sequence as set forth in any of Tables 1-4.

14. The method of any one of claims 1-13, wherein the at least one RAN protein is encoded by a gene selected from Table 6.

15. The method of any one of claims 1-13, wherein the at least one RAN protein is encoded by Rab20 or ADAMTS14.

16. The method of any one of claims 1-15, wherein the number of poly amino acid repeats in the at least one RAN protein is at least 40.

17. The method of any one of claims 1-16, wherein an antigen retrieval method is performed on the biological sample prior to the detecting.

18. The method of any one of claims 7-17, wherein the therapeutic agent comprises a small molecule, interfering nucleic acid, DNA aptamer, RNA aptamer, protein, or antibody.

19. The method of claim 18, wherein the small molecule comprises an inhibitor of eukaryotic initiation factor 2 (eIF2), eukaryotic initiation factor 3 (eIF3), protein kinase R (PKR), p62, LC3 I subunit, LC3 II subunit, TARBP2, or Toll-like receptor 3 (TLR3).

20. The method of claim 18 or claim 19, wherein the small molecule comprises metformin or a pharmaceutically acceptable salt, co-crystal, tautomer, stereoisomer, solvate, hydrate, polymorph, isotopically enriched derivative, or prodrug thereof; buformin; or phenformin.

21. The method of claim 18, wherein the interfering nucleic acid comprises a dsRNA, siRNA, shRNA, miRNA, artificial miRNA (ami-RNA), or antisense oligonucleotide (ASO).

22. The method of claim 18 or claim 21, wherein the interfering nucleic acid inhibits expression of eukaryotic initiation factor 2 (eIF2), eukaryotic initiation factor 3 (eIF3), protein kinase R (PKR), p62, LC3 I subunit, LC3 II subunit, Toll-like receptor 3 (TLR3), a gene comprising a nucleic acid sequence as set forth in any one of Tables 1-4, or a gene selected from Table 6.

23. The method of claim 18, claim 21, or claim 22, wherein the interfering nucleic acid inhibits expression of one or more eIF3 subunits selected from the group consisting of eIF3a, eIF3b, eIF3c, eIF3d, eIF3e, eIF3f, eIF3g, eIF3h, eIF3i, eIF3j, eIF3k, eIF3l, and eIF3m.

24. The method of claim 18, claim 21, or claim 22, wherein the interfering nucleic acid comprises a region of complementarity with any one of the nucleic acid sequences set forth in Tables 1-4.

25. The method of claim 18, wherein the protein inhibits eIF2, eIF3, PKR, p62, LC3 I subunit, LC3 II subunit, TLR3, a gene comprising a nucleic acid sequence set as forth in any one of Tables 1-4, or a gene selected from Table 6.

26. The method of claim 18 or claim 25, wherein the protein is a dominant-negative variant of PKR.

27. The method of claim 26, wherein the dominant-negative variant comprises a mutation at amino acid position 296, optionally wherein the mutation is K296R.

28. The method of any one of claim 18 or 25-27, wherein the protein is delivered to the subject by a vector.

29. The method of claim 28, wherein the vector is a viral vector, optionally a recombinant adeno-associated virus (rAAV).

30. The method of claim 29, wherein the rAAV comprises an AAV9 capsid protein or variant thereof.

31. The method of claim 18, wherein the antibody targets eIF2, eIF3, PKR, p62, LC3 I subunit, LC3 II subunit, TLR3, or one or more RAN proteins.

32. The method of claim 31, wherein the one or more RAN proteins is a poly(GR), poly(GP), poly(PR), and/or poly(GA) RAN protein(s).

33. The method of claim 31 or claim 32, wherein the antibody specifically binds to the poly-amino acid repeat of the one or more RAN protein(s).

34. The method of claim 31 or claim 32, wherein the antibody specifically binds to the C-terminus of the one or more RAN protein(s).

35. The method of any one of claim 18 or 31-34, wherein the antibody is a monoclonal antibody or a polyclonal antibody.

36. The method of any one of claims 18-35, wherein the therapeutic agent inhibits translation of one or more RAN proteins.

37. The method of any one of claims 7-36, further comprising administering a second therapeutic agent to the subject.

38. The method of claim 37, wherein the second therapeutic agent is selected from donepezil, galantamine, memantine, rivastigimine, or a combination thereof.

39. The method of any one of claims 1-38, wherein the detecting is performed by dot blot, binding assay, hybridization assay, immunoblot analysis, 2-D gel electrophoresis, Western blot, immunohistochemistry (IHC), ELISA, RCA-based ELISA, rtPCR-based ELISA, label free immunoassays such as surface plasmon resonance bio layer interferometry, immunoquantitative PCR, mass spectrometry such as GC-MS, LC-MS, MALDI-TOF-MS, bead based immunoassays, immunoprecipitation, immunostaining, or immunoelectrophoresis.

40. The method of claim 39, wherein the ELISA is RCA-based ELISA or rtPCR-based ELISA.

41. The method of claim 39, wherein the Western blot comprises contacting the sample with an anti-RAN antibody, wherein the anti-RAN antibody targets:
(i) a poly(GP), poly(GR), poly(PR), and/or poly(GA) repeat region of a RAN protein; or
(ii) the C-terminus of a RAN protein that comprises an amino acid sequence that is not the repeat amino acid sequences poly(GP), poly(GA), poly(GR), or poly(PR).

42. The method of claim 39, wherein the hybridization assay comprises Fluorescence In situ Hybridization (FISH) and/or dCas9-based enrichment.

43. The method of claim 42, wherein the dCas9-based enrichment is performed using a Streptococcus pyogenes dCas9 (spdCas9).

44. The method of claim 42, wherein the dCas9-based enrichment is performed using a Cas9 protein that is a mutant of a wild-type Cas9.

45. The method of any one of claims 42-44, wherein the dCas9-based enrichment is performed using a Cas9 protein that comprises a mutation that inactivates a Cas9 nuclease activity.

46. The method of any one of claims 42-45, wherein the dCas9 protein comprises a Staphylococcus aureus dCas9, a Streptococcus pyogenes dCas9, a Campylobacter jejuni dCas9, a Corynebacterium diphtheria dCas9, a Eubacterium ventriosum dCas9, a Streptococcus pasteurianus dCas9, a Lactobacillus farciminis dCas9, a Sphaerochaeta globus dCas9, an Azospirillum dCas9, a Gluconacetobacter diazotrophicus dCas9, a Neisseria cinerea dCas9, a Roseburia intestinalis dCas9, a Parvibaculum lavamentivorans dCas9, a Nitratifractor salsuginis dCas9, a Campylobacter lari dCas9, or a Streptococcus thermophilus dCas9.

47. The method of any one of claims 42-46, wherein the detecting comprises contacting the sample with an anti-RAN antibody.

48. The method of claim 47, wherein the anti-RAN protein antibody targets:
(i) a poly(GP), poly(GR), poly(PR), and/or poly(GA) repeat region of a RAN protein; or
(ii) the C-terminus of a RAN protein that comprises an amino acid sequence that is not the repeat amino acid sequences poly(GP), poly(GA), poly(GR), or poly(PR).

49. The method of any one of claims 42-48, wherein the detecting further comprises nucleic acid sequencing, optionally wherein the sequencing is Next-Generation Sequencing (NGS).

50. A method of monitoring a C9orf72 negative sporadic amyotrophic lateral sclerosis (C9− sALS) therapeutic regimen, the method comprising:
(i) detecting in a second biological sample obtained from a subject that has been administered a therapeutic regimen for C9− sALS a level of one or more poly(GR), poly(GP), poly(GA), and/or poly(PR) repeat-associated non-ATG (RAN) protein(s);
(ii) comparing the level of one or more RAN proteins detected in (i) to a level of the same RAN protein(s) in a first biological sample obtained from the subject prior to being administered the therapeutic regimen; and
(iii) continuing to administer the therapeutic regimen for C9− sALS when the level of the one or more RAN protein(s) in the second biological sample is reduced compared to the level of the first biological sample,
wherein the first biological sample and/or the second biological sample is blood, serum or cerebrospinal fluid (CSF).

51. A therapeutic agent for the treatment of C9orf72 negative sporadic amyotrophic lateral sclerosis (C9− sALS), wherein the therapeutic agent is a small molecule, interfering nucleic acid, DNA aptamer, RNA aptamer, protein, or antibody that reduces expression of one or more poly(GR), poly(GP), poly(GA), and/or poly(PR) repeat-associated non-ATG (RAN) protein(s).
